(19) United States
(12) Patent Application Publication
(10) Pub. No.: US 2022/0041981 A1
(43) Pub. Date: Feb. 10, 2022

(54) MULTIPOTENT ADULT STEM CELLS: CHARACTERIZATION AND USE

(71) Applicant: New York Medical College, Valhalla, NY (US)

(72) Inventors: Paul LUCAS, Valhalla, NY (US); Jessica C. BLACK, Valhalla, NY (US)

(21) Appl. No.: 17/408,272

(22) Filed: Aug. 20, 2021

Related U.S. Application Data
(63) Continuation of application No. 16/206,297, filed on Nov. 30, 2018.
(60) Provisional application No. 62/592,957, filed on Nov. 30, 2017.

(51) Int. Cl. C12N 5/074; A61K 35/545

(52) U.S. Cl. CPC C12N 5/0607; A61K 35/545

(57) ABSTRACT

The present invention relates to biomarkers, methods, and compositions for characterizing and isolating multipotent adult stem cells (MASCs) and uses thereof.

This application claims priority to U.S. Provisional Application No. 62/592,957, filed on Nov. 30, 2017, the contents of which are hereby incorporated by reference in their entirety.

### FIELD OF THE INVENTION

The present invention relates to biomarkers, methods, and compositions for characterizing and isolating multipotent adult stem cells (MASCs) and uses thereof.

### BACKGROUND

Stem cells are cells that are 1) capable of self-renewal (proliferate while maintaining an undifferentiated state) and 2) differentiate into at least one phenotype[15]. Thus, a stem cell is a special kind of cell that has a unique capacity to renew itself and to give rise to specialized cell types. Although most cells of the body such as heart cells or skin cells are committed to conduct a specific function, a stem cell is uncommitted and remains uncommitted, until it receives a signal to develop into a specialized cell. In 1998, stem cells from early human embryos were first isolated and grown in culture. It is recognized that these stem cells, called embryonic stem cells, are, indeed, capable of becoming almost all of the specialized cells of the body. In recent years, stem cells present in adults also have been shown to have the potential to generate replacement cells for a broad array of tissues and organs, such as the heart, the liver, the pancreas, and the nervous system.

Different varieties of adult stem cells include mesenchymal stem cells (MSCs), hematopoietic stem cells (HSCs), and neural stem cells (NSCs). Apart from hematopoietic stem cells, mesenchymal stem cells are the most commonly studied adult stem cells. MSCs are defined by their plastic adherence, positivity for CD 73, CD 90, and CD 105, negativity for CD45, CD34, CD14 or CD11b, HLA-DR, and ability to differentiate to adipocytes, chondrocytes, and osteoblasts in vitro [2]. MSCs also secrete a large number of cytokines that induce cellular proliferation and modulate the immune system. MSCs were first isolated from bone marrow [6], but since then have been isolated from a number of different tissues including, adipose [7], umbilical cord blood [7], dental pulp [8] and placenta [8a]. Each tissue that an MSC can be isolated from is considered to be a family within MSCs. Thus, there exists a bone-marrow family of MSCs and a placental family of MSCs.

While the use of adult MSCs in regenerative research is commonplace today, there are a number of reasons that they are not ideal for regenerative medicine. One drawback of using adult MSCs within stem cell research is their limited proliferation potential; they have a limited expansion capacity. While adult MSCs can be expanded in vitro, over time, they lose proliferation, differentiation, and immunomodulation [3,4]. Ex vivo, expansion of MSCs alters their ability to repair double strand DNA breaks, which is necessary for cells intended for transplantation [12]. Adult MSCs are also usually donor derived, which is not ideal when conducting research due to a loss of efficiency in cell culture methods. Better alternatives to adult MSCs are needed. The efficacy of embryonic MSCs has been evaluated and preliminary results indicate that they reduce certain problems in proliferation and derivation [5]. However, there are limitations associated with obtaining embryonic MSCs. In addition, there is growing speculation amongst MSC scientists that MSCs are not, in fact, even stem cells and rarely differentiate in vivo. In this case, their applicability toward regenerative gem cell research is called into question. Thus, there is a need for alternatives to MSC's for regenerative stem cell research and also for related therapeutic options.

### SUMMARY OF THE INVENTION

In certain embodiments, the present invention relates to isolated multipotent adult stem cells (MASCs), expressing two or more genes of Table 4, Table 5, and/or Table 10. In additional embodiments, the MASCs express five or more genes of Table 4, Table 5, and/or Table 1.0. In additional embodiments, the MASCs express ten or more genes of Table 4, Table 5, and/or Table 10.

In additional embodiments, the MASCs express fifteen or more genes of Table 5 and/or Table 10. In additional embodiments, the MASCs express EREG (epiregulin). In additional embodiments, the MASCs express SPINK6 and/or ROS1.

In yet additional embodiments, the MASCs do not express Oct-4, Sox-2, and Nanog. In additional embodiments, the MASCs do not form teratomas in vivo. In yet further embodiments, the MASCs are isolated from human foreskin.

In certain embodiments, the present invention. relates to a composition comprising the MAC cells described herein.

In yet additional embodiments, the present invention relates to a biodegradable matrix comprising the MASCs described herein.

In yet additional embodiments, the present invention relates to a kit comprising the MASCs described herein.

In certain embodiments, the kit comprises antibodies specific to proteins expressed by two or more genes of Table 4, Table 5, and/or Table 10.

In yet additional embodiments, the present invention relates to a method Of isolating multipotent adult stem cells (MASCs), comprising selecting markers expressed by two or more genes of Table 4, Table 5, and/or Table 10.

In yet additional embodiments, the present invention relates to a method for restoring tissue or improving, wound healing in a patient in need thereof comprising administering an effective amount of the composition of MASCs described herein to the patient.

In yet additional embodiments, the present invention relates to a method for regenerating or repairing tissue in a patient in need thereof, comprising administering an effective amount of the composition of MASCs described herein to the patient.

In certain embodiments, the tissue comprises skin, bone, meniscus, cartilage or any combination thereof.

### BRIEF DESCRIPTION OF THE DRAWINGS

The patent or application file contains at least one drawings executed in color. Copies of this patent or patent application publication with color drawing(s) will be provided by the Office upon request and payment of the necessary fee.

FIG. 1 is a Venn Diagram showing gene expression profiles in MASCs, BM-MSCs, and PL-MSCs.

FIG. 2 shows the functional annotation clustering results of the DAVID software using the gene list of 1,014 genes found to be only expressed in Hfs when compared with ESCs.

FIG. 3 shows the gene report for Functional Annotation Cluster 1, showing that SPINK6, SERPINB10, and SERPINB2 are within the cluster.

FIG. 4 shows DAVID functional annotation clustering gene report for Cluster #4.

FIGS. 5A-5D are images showing implantation site. FIGS. 5A-C, injection site with 42×10⁶ cells injected in 0.3 ml of Dulbecco's Medium without phenol red. FIG. 5A. Day 0, FIG. 5B. Day 2, FIG. 5C. Day 7. FIG. 5D. Implantation of PGA+HfSCs 4 weeks post-implantation.

FIGS. 6A-6D are images showing human MASCs within PGA FIG. 6A. Human MASCs in PGA prior to implantation, Toluidine Blue stained. Red arrow points to PGA. Yellow arrow points to cells. FIG. 6B. Immunostained human MASCs in PGA prior to implantation. Section stained with antibody to human gamma-actin with green secondary. Blue arrows point to PGA. Yellow arrow points to cells. FIG. 6C. PGA+MASCs 4 weeks post-implantation. Toluidine Blue stained. Red arrow points to PGA and yellow arrow points to cells. FIG. 6D, PGA+MASCs 4 weeks post-implantation immunostained using pan-specific antibody to gamma action (red secondary) and antibody specific to human gamma-actin (green secondary). Blue arrows point to PGA. Yellow arrows point to cells double labeled with both antibodies, indicating human MASCs.

FIGS. 7A-7F are photomicrographs of the injection site. FIG. 7A. Rat MASCs stained with Toluidine Blue, The needle track (NT) is clearly visible. FIG. 7B. Immunohistochemistry of rat MASCs in A. Red=pan specific antibody to gamma actin. Green=human specific antibody to gamma actin. Red arrows point to rat MASCs. FIGS. 7C and 7E. Human MASCs in needle track stained with Toluidine Blue. FIG. 7C is animal X22 and FIG. 7E is animal X26. M=skeletal, muscle. FIGS. 7D and 7F. Immunohistochemistry of human MASCs in FIGS. 7C and 7E. Red=pan specific antibody to gamma actin. Green=human specific antibody to gamma actin. Yellow arrows point to cells double stained with both antibodies. Green arrow points to cells mostly stained with antibody to human gamma actin. Red arrows point to host cells.

FIGS. 8A-8D are images of rat bone marrow. FIG. 8A. Rat bone marrow stained with an antibody to CD45 with green secondary antibody. b=bone. Green arrow points to CD45+ cells in marrow. FIGS. 8B-D. injection sites of X22, X24, and X27, respectively stained with antibody to CD4 with green secondary and antibody to human-specific gamma-actin with red secondary.

FIGS. 9A-9F are photomicrographs of sections near the injection site of human MASCs 4 weeks post-injection. FIG. 9A. Toluidine blue stained section of animal X23. Black arrow+points to blood vessel. FIG. 913. Same vessel stained with antibodies to pan-specific gamma actin (red) and human specific gamma actin (green). Yellow arrow points to the same blood vessel, which stains positive for both antibodies to gamma actin, indicating human origin. FIG. 9C. Blood vessel in animal X24 stained with both anti-gamma-actin antibodies. Red arrow points to cells stained only red (pan specific) while yellow arrow points to cells stained with both antibodies (human), FIG. 90. Animal X25 sectioned stained with antibody to human CD31. White arrow points to negative stained (host) blood vessel. Green arrows point to positive stained (donor) blood vessels. FIG. 9E. Animal. X26 stained with antibody to smooth muscle alpha actin (red) and human gamma actin (green). Yellow arrows point to blood vessels positive for both. FIG. 9F. Animal X22 stained with antibody to desmin (red) and human gamma actin (green). Yellow arrow points to vessel positive for both.

FIGS. 10A-10F are photomicrographs of sections near the injection site of human MASCs 4 weeks post-injection. FIG. 10A. Hematoxylin-eosin stained hair follicle (HF) in animal X24. FIG. 10B. Immunohistochemistry in adjacent slide stained with antibodies to pan-specific gamma actin (red) and human specific gamma actin (green). Yellow arrow points to the hair follicle. FIG. 10C. Immunohistochemistry of hair follicles in animal X26 stained with antibody to keratinocytes (red) and human specific gamma actin (green). Yellow arrows point to cells positive for both antibodies. FIG. 10D. Immunohistochemistry of hair follicles in animal X27 stained with antibody to keratinocytes (red) and human specific gamma actin (green). Yellow arrows point to cells positive for both antibodies. G=apparent gland. FIG. 10E. Immunohistochemistry of tissue that appears to be a gland in animal X26 stained with antibodies to pan-specific gamma actin (red) and human specific gamma actin (green). Green arrows point to cells positive for anti-human gamma actin. Yellow arrow points to cells positive for both antibodies. FIG. 10F. Immunohistochemistry of tissue that appears to be a gland in animal X23 stained with antibodies to pan-specific gamma actin (red) and human specific gamma actin (green). Yellow arrow points to cells positive for both antibodies.

FIG. 11 shows a gene expression profile of genes in development lineage across 5 cells types. This is a heat map of changes in expression between significantly expressed genes from pathways of interest including housekeeping and cell cycle between all 5 cell types. Unregulated expression is indicated in shades toward red, downregulated expression in shades toward blue. Equal expression is white. MASC=multipotent adult stem cells. ESC embryonic stein cells. SMC=smooth muscle cells. BM-MSC=bone marrow-derived mesenchymal stem cells. PL=placental derived mesenchymal stem cells. individual genes are listed on the y-axis on the right side. All 5 cell types have unique expression profiles for the genes tested. However, BM-MSC and PL-MSC do have overlapping expression profiles and are somewhat similar to each other.

FIG. 12 shows a gene expression profile of miRNAs across 5 cell types. This is a heat map of changes in expression of microRNAs between the 4 cell types. Upregulated expression is indicated in shades toward red, downregulated expression in shades toward blue. Equal expression is white. MASC=multipotent adult stem cells. SMC smooth muscle cells. ESC=embryonic stem cells. PL=placental derived mesenchymal stem cells BM-MSC=bone marrow-derived mesenchymal stem cells. Individual miRNAs are listed on the y-axis on the right side. Each cell type has a unique expression profile of miRNAs. In particular, MASCs are unique from the other 4 cell types. PL and BM-MSCs are somewhat similar but unique from MASCs, SMCs, and ESCs.

FIG. 13 shows SNORA expression of all 5 cell types. This is a heat map of changes in expression of SNORAs expressed by any of the S cell types. Upregulated expression is indicated in shades toward red, downregulated expression in shades toward blue. Equal expression is is white. ESC=embryonic stem cells. MASC=multipotent adult stem cells. SMC=smooth muscle cells. BM-MSC=bone marrow-derived mesenchymal stem cells. PL placental derived mesenchymal stem cells. Individual SNORAs are listed on the y-axis on the right side. SNORAs are small nucleolar RNAs, H/ACA box family, They are considered part of the epigenetic profile. ESCs are distinct from all the other cell types. BM and PL-MSCs have similar, but not identical, expression patterns. MASCs and SMCs have similar, but not identical, expression profiles.

FIG. 14 shows SNORD expression of all 5 cell types. This is a heat map of changes in expression of SNORDs expressed by any of the 5 cell types. Upregulated expression is indicated in shades toward red, downregulated expression in shades toward blue. Equal expression is white. MASC=multipotent adult stem cells. SMC=smooth muscle cells. BM-MSC=bone marrow-derived mesenchymal stem cells. ESC=embryonic stem cells. PL=placental derived mesenchymal stem cells. Individual SNORDs are listed on the y-axis on the right side. SNORDs are small nucleolar RNAs, CD box gene family. They are considered part of the epigenetic profile. MASCs have a nearly identical expression profile to SMCS but different from ESCs and the two families of MSCs.

FIG. 15 shows PCA analysis between all cell types. PCA analysis based on the significantly expressed genes by MASCs. ESCs=embryonic stem cells. HfSC MASCs=human MASCs. SMC=smooth muscle cells. PL-MSC placental derived mesenchymal stem cells. BM-MSCs=bone marrow derived mesenchymal stem cells. The embryonic stem cells (ESCs) are located away from all the other cell types. MASCs are located distant from the ESCs and both families of MSCs. MASCs are located closest to SMCs.

FIGS. 16A-16B show full thickness skin defects in rats immediately post-op. FIG. 16A empty defect. FIG. 168 defect with PGA+MASCs.

FIGS. 17A-17B show immunohistochemistry 8 weeks post-op for the defects depicted in FIGS. 16A-16B. Sections were stained with an antibody to keratinocytes (red), human MASCs (green), and nuclei (blue). FIG. 17A=Empty defect. FIG. 17B=defect treated with PGA+human MASCs. Red arrow points to unorganized keratinocytes on the surface of the scar tissue in the empty defect. Yellow arrow points to normal epidermis (keratinocytes) stained both for red keratinocytes and green MASCs (merge is yellow). Green arrow points to green cells (MASCs) in the dermis. White arrow points to hair follicle emerging from the epidermis.

FIGS. 18A-18B show immunohistochemistry of the dermal, defect treated with PGA+human MASCs 8 weeks post-op. FIG. 18A. Section stained with an antibody to keratinocytes (red), antibody to human gamma-actin (green) and DAPI for nuclei (blue). Yellow arrow points to hair leading to hair follicle to the left. Green arrow points to glandular duct, with the eccrine gland to the left. FIG. 18B. Section stained with the antibody to human gamma actin (green), glands (GDC-FP15, red), and DAN for nuclei (blue). Orange arrows point to eccrine glands which stain orange/yellow. FIG. 18A shows a picture of a hair follicle and a gland and duct within the defect treated with PGA+MASCs. The cells lining the duct and the cells of the gland both stain green for human gamma actin protein, indicating that they are derived from the human MASCs. FIG. 18B shows a section stained with an antibody, GCD-FP15, an antibody for eccrine sweat glands (www.researchgate.net/publication/275949302_Gross_Cystic_Disease_Fluid_Protein_15_in_Str atum_Corneum_Is_a_Potential_Marker_of_Decreased_Eccrine_Sweating_for_Atopic_Dermatiti s) and human gamma actin. The glands appear orange (yellow+red) indicating the gland cells are human in origin.

FIGS. 19A-19B show the detrital defect treated with PGA+human MASCs 8 weeks post-op stained for endothelial cells. Sections stained with an antibody to human CD31 for endothelial cells (green) and DAPI stain for nuclei (blue). A. Green arrows point to blood vessels with cells positive for human CD31. White arrows point to blood vessels negative for human CD31 (rat origin). B. A larger vein. The endothelial cells (green arrows) are positive for human CD31.

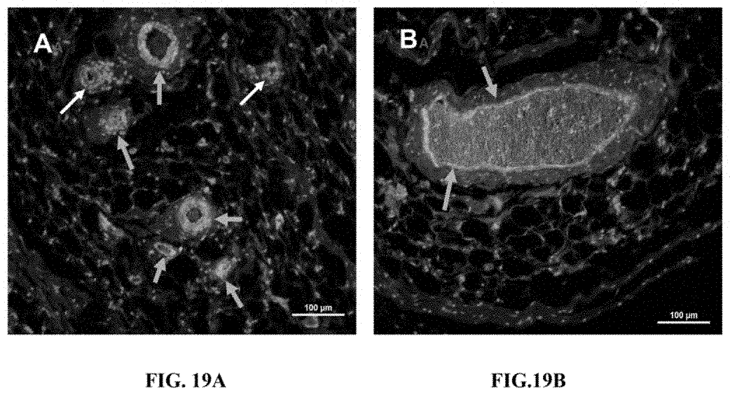

Red blood cells can be discerned inside the vessel. Sections were stained with an antibody specific for human CD31, a marker for endothelial cells. FIG. 19A. shows several blood vessels in the dermis (green arrows) that have endothelial cells positive for human CD31, indicating these cells differentiated from the human MASCs. The white arrows show two vessels that are negative for human CD31, indicating these are host vessels. A larger vein is shown in FIG. 19B. The endothelial cells are positive for human CD31. There are red and white blood cells within the vein, indicating that it is functional.

### DETAILED DESCRIPTION

A unique population of adult stem cells were isolated and were termed multipotent adult stem cells (MASCs) and served as the basis for the present studies. MASCs are undifferentiated cells found in several tissues in post-natal animals. As shown in the data herein, the transcriptome of several novel Multipotent Adult Stem Cells (MASCs) is characterized. We compare MASCs to currently used stem cells in regenerative Medicine research, Mesenchymal Stem Cells (MSCs). Using RNA-seq in biological triplicate, the transcriptomes of each cell type were derived. The transcriptomes of Hf-derived MASCs, bone-marrow-derived (BM) and placental-derived (PL) mesenchymal stem cells were compared to each other and analyzed.

MASCs have, when implanted into animal models in an undifferentiated state, regenerated several tissues, apparently by responding to local cues in vivo to differentiate into tissues at the site. To date, very little is known about the gene expression of profile of MASCs. In contrast to MSCs, Multipotent adult stem cells (MASCs) have an apparent unlimited proliferation potential in vitro in the undifferentiated state. They have also been shown to have the ability to generate progeny of several distinct cell types of all three dermal lineages in culture. These phenotypes include, but are not limited to, chondrocytes, osteoblasts, and adipocytes. In vivo, MASCs are able to respond to local cues for differentiation into tissue at specific sites and regenerate tissues due to their differentiation, but are not able to form tumors. Unlike ESCs (embryonic stem cells), MASCs do not spontaneously differentiate in culture, do not express Oct-4, Sox-2, and Nanog, and do not form teratomas in vivo. Thus MASCs are not identical to either MSCs or ESCs which are both commonplace standards in the field of research regarding regeneration technology.

By way of definition, the following terms are understood in the art: A “stem cell” is a cell from the embryo, fetus, or adult that has, under certain conditions, the ability to reproduce itself for long periods or, in the case of adult stem cells, throughout the life of the organism. It also can give rise to specialized cells that make up the tissues and organs of the body.

A “pluripotent stem cell” has the ability to give rise to types of cells that develop from the three germ layers (mesoderm, endoderm, and ectoderm) from which all the cells of the body arise. The only known sources of human pluripotent stem cells are those isolated and cultured from early human embryos and from fetal tissue that was destined to be part of the gonads.

An “embryonic stem cell” is derived from a group of cells called the inner cell mass, which is part of the early (4- to 5-day) embryo called the blastocyst. Once removed from the blastocyst the cells of the inner cell mass can be cultured into embryonic stem cells. These embryonic stem cells are not themselves embryos.

An “adult stem cell” is an undifferentiated (unspecialized) cell that occurs in a differentiated (specialized) tissue, renews itself, and becomes specialized to yield all of the specialized cell types of the tissue in which it is placed when transferred to the appropriate tissue. Adult stem cells are capable of making identical copies of themselves for the lifetime of the organism. This property is referred to as “self-renewal.” Adult stem cells usually divide to generate progenitor or precursor cells, which then differentiate or develop into “mature” cell types that have characteristic shapes and specialized functions, e.g., muscle cell contraction or nerve cell signaling. Sources of adult stem cells include bone marrow, blood, the cornea and the retina of the eye, brain, skeletal muscle, dental pulp, liver, skin, the lining of the gastrointestinal. tract and pancreas.

Stem cells from the bone marrow are the most-studied. type of adult stem cells. Currently, they are used clinically to restore various blood and immune components to the bone marrow via transplantation. There are currently identified two major types of stem cells found in bone marrow: hematopoietic stem cells (HSC, or CD34+ cells) which are typically considered to form blood and immune cells, and stromal (mesenchymal) stem cells (MSC) that are typically considered to form bone, cartilage, muscle and fat, However, both types of marrow-derived stem cells recently have demonstrated extensive plasticity and multipotency in their ability to form the same tissues.

A “progenitor or precursor” cell occurs in fetal or adult tissues and is partially specialized; it divides and gives rise to differentiated cells. Researchers often distinguish precursor/progenitor cells from adult stem cells in that when a stem cell divides, one of the two new cells is often a stem cell capable of replicating itself again. In contrast when a progenitor/precursor cell divides, it can form more progenitor/precursor cells or it can form two specialized cells. Progenitor/precursor cells can replace cells that are damaged or dead, thus maintaining the integrity and functions of a tissue such as liver or brain.

General means for isolating and culturing stem cells useful in the present invention are well known. Umbilical cord blood is an abundant source of hematopoietic stem cells. The stem cells obtained from umbilical cord blood and those obtained from bone marrow or peripheral blood appear to be very similar for transplantation use [Inaba et al., J. Exp. Med. 176:1693-1702(1992); Ho et al., Stem Cells 13 (suppl. 3): 100-105(1995); Brenner, Journal of Hematotherapy 2: 7-17 (1993)]. However, these particular stem cells cannot be isolated or grown using the methods here. Placenta is an excellent readily available source for mesenchymal stem cells. Moreover, mesenchymal stem cells have been shown to be derivable from adipose tissue and bone marrow stromal cells and speculated to be present in other tissues. Methods for isolating, purifying and culturally expanding mesenchymal stem cells are known. Specific antigens for MSC are also known. (see, U.S. Pat. Nos. 5,486,359 and 5,837,539).

### Pharmaceutical Compositions

In other embodiments, the present invention provides pharmaceutical compositions comprising MASC cells and optionally any acceptable excipients. In other aspects, the present invention features kits for treating tissue damage. Stem cells generally have been presented to the desired organs either by injection into the tissue, by infusion into the local circulation, or by Mobilization of autologous stem cells from the marrow accompanied by prior removal of stem cell-entrapping organs before mobilization when feasible, i.e., splenectomy.

The MASCs described herein may be administered in any suitable manner, preferably with pharmaceutically acceptable carriers. Suitable methods of administering such cells to a patient are available, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention.

Formulations suitable for parenteral administration, such as, for example, by intraarticular (in the joints), intravenous, intramuscular, intradermal, intraperitoneal, and subcutaneous routes, include, aqueous and non-aqueous, isotonic sterile injection. solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Parenteral administration is one useful method of administration. The formulations can be presented in unit-dose or multi-dose sealed containers, such as ampules and vials, and in some embodiments, can be stored in a freeze-dried (lyophilized) condition requiring only the addition of the sterile liquid carrier, for example, saline, for injections, immediately prior to use. These formulations may be administered with factors that. mobilize the desired class of adult stem cells into the circulation.

Extemporaneous injection solutions and suspensions can be prepared frond sterile powders, granules, and tablets of the kind previously described. Cells transduced by the vector as described above in the context of ex vivo therapy can also be administered parenterally as described above, except that lyophilization is not generally appropriate, since cells are destroyed by lyophilization.

The dose administered to a patient, in the context of the present invention should, be sufficient to effect a beneficial therapeutic, response in the patient over time. The dose will be determined by the efficacy of the particular cells employed and the condition of the patient, as well as the body weight of the patient to be treated. The size of the dose also will be determined by the existence, nature, and extent of any adverse side effects that accompany the administration of a cell type in a particular patient.

For administration, cells of the present invention can be administered at a rate determined by the LD-50 of the cell type, and the side effects of the cell type at various concentrations, as applied to the mass and overall health of the patient. Administration can be accomplished via single or divided doses. Adult stem cells may also be mobilized using exogenously administered factors that stimulate their production and egress from tissues or spaces, that may include, but are not restricted to, bone marrow or adipose tissues.

A “prophylactically effective amount” refers to an amount effective, at dosages and far periods of time necessary, to achieve the desired prophylactic result. Typically, since a prophylactic dose is used in subjects prior to or at an earlier stage of disease, the prophylactically effective amount will be less than the therapeutically effective amount.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

The term “defect” as used herein refers to an imperfection that impairs worth or utility or the absence of something necessary for completeness or perfection; or a deficiency in function. The term defect as used herein is not limited to acquired defects, for example defects from damage from, for example, diseases such as osteoarthritis or arthritis, injury or wear, but the term defects also encompasses defects due to non-acquired or existing defects, for example congenital or developmental defects.

As defined herein, an “osteochondral defect” is a focal area of articular damage with cartilage damage and injury of the adjacent subchondral bone and may be due to Osteochondritis dissecans (OCD), avascular necrosis, or trauma. In a particular embodiment said osteochondral defect is due to osteoarthritis or alternatively, joint trauma.

As used herein, the term “articular cartilage”, is understood to mean any cartilage tissue, that biochemically and morphologically resembles the cartilage normally found on the articulating surfaces of mammalian joints.

As used herein, the term “polymer” in the present application is intended to mean without limitation a polymer solution, polymer suspension, a polymer particulate or powder and a polymer micellar suspension.

As used herein, the term “bioresorbable” refers to the ability of a material to be reabsorbed in vivo. The absorbable polymer material can is selected. from the group consisting of polyglycolic acid (PGA), polylactic acid (PLA), polylactic-co-glycolic) acid (PLGA), polyanhydride, polycapralactone (PCL), polydioxanone and polyorthoester. The bioabsorbable polymer material also can be composite material that comprises an absorbable polymer material and other materials.

As used herein, the term “biodegradable” as used herein denotes a composition that is not biologically harmful and can be chemically degraded or decomposed by natural effectors (e.g., weather, soil bacteria, plants, animals).

As used herein, the term “glycolide” is understood to include polyglycolic acid. Further, the term “lactide” is understood to include L-lactide, D-lactide, blends thereof, and lactic acid polymers and copolymers.

The term “polyglycolic acid”, “poly(glycolic) acid and “PGA” are used interchangeably herein, refer to a polymer of glycolic acid. The term “polylactic acid”, “poly(lactic) acid” and “PLA” are used interchangeably herein, refer to a member of the polyester family, in particular the poly(α-hydroxyl acid) family, and refers to a polymer of lactic acid molecules. The terms PLA and polylactic acid are intended to encompass all isometric forms of poly(lactic)acid, for example d(), l(+) and racemic (d,l) and the polymers are usually abbreviated to indicate the chirality. Poly(I)LA and poly(d)LA are semi-crystalline solids.

### Polymeric Material

In certain embodiments, the MASCs described herein can be utilized in conjunction with a polymeric material. Such polymeric material can include porous material, including but not limited to polymeric mesh or sponge. The polymeric material can in a specific embodiment, may be in the form of felt. In another particular embodiment, the polymeric material is biodegradable over a time period of between about two weeks to about two years. The polymeric material can be manufactured or constructed using commercially available materials. This material is typically derived from a natural or a synthetic polymer Biodegradable polymers are preferred, so that the newly formed tissue can maintain itself and function normally without the extraneous material of the polymer. Synthetic polymers are preferred because their degradation rate can be more accurately determined and they have more lot to lot consistency and less immunogenicity than natural polymers. Natural polymers that can be used include but ate not limited, to proteins such as collagen, albumin, and fibrin; and polysaccharides such as alginate and polymers of hyaluronic acid. Synthetic polymers include both biodegradable and non-biodegradable polymers. Examples of biodegradable polymers include but are not limited to polymers of hydroxyl acids such as polylactic acid (PLA), polyglycolic acid (PGA), and polylactic acid-glycolic acid (PLGA), polyorthoesters, polyanhydrides, polyphosphazenes, polydioxanone, those described in WO2007022149 and US20070036842, GELFOAM® polycaprone. Polymeric materials used in the instant method may be obtained from commercial sources such as Biomedical Structures, Inc., Ethicon, or Pfizer, and combinations thereof. Non-biodegradable polymers include polyacrylates, polymethacrylates, ethylene vinyl acetate, and polyvinyl alcohols. These should be avoided since their presence in the tissue will inevitably lead to areas where the natural tissue is not restored.

The starting material may, in a specific embodiment, include a bioabsorbable such as polyglycolic acid, or poly-L lactide or copolythers that include one of each. The polymer composition, as well as method of manufacture, can be used to determine the rate of degradation. For example, mixing increasing amounts of polyglycolic acid with polylactic acid decreases the degradation time.

The term “subject” as used in this application means an animal with an immune system such as avians and mammals. Mammals include canines, felines, rodents, bovine, equines, porcines, ovines, primates, and humans. Avians include, but are not limited to, fowls, songbirds, and raptors. Thus, the invention can be used in veterinary medicine, e.g., to treat companion animals, farm animals, laboratory animals in zoological parks, and animals in the wild. The invention is particularly desirable for human medical applications.

The term “patient” as used in this application means a human subject. The terms “treat”, “treatment”, and the like refer to a means to slow down, relieve, ameliorate or alleviate at least one of the symptoms of the disease, or reverse the disease after its onset.

The terms “prevent”, “prevention”, and the like refer to acting prior to overt disease onset, to prevent the disease from developing or minimize the extent of the disease or slow its course of development.

The term “agent” as used herein means a substance that produces or is capable of producing an effect and would include, but is not limited to, chemicals, pharmaceuticals, biologics, small organic molecules, antibodies, nucleic acids, peptides, and proteins.

The phrase “therapeutically effective amount” is used. herein to mean an amount sufficient to cause an improvement in a clinically Significant condition in the subject, or delays or minimizes or mitigates one or more symptoms associated with the disease, or results in desired beneficial change of physiology in the subject.

As used herein, the term “isolated” and the like means that the referenced material is free of components found in the natural environment in which the material is normally found. In particular, isolated biological material is free of cellular components. In the case of nucleic acid molecules, an isolated nucleic acid includes a PCR product, an isolated mRNA, a cDNA, an isolated genomic DNA, or a restriction fragment. In another embodiment, an isolated nucleic acid is preferably excised from the chromosome in which it may be found. Isolated nucleic acid molecules can be inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated material may be, but need not be, purified.

The term “purified” and the like as used herein refers to material that has been isolated under conditions that reduce or eliminate unrelated materials, i.e., contaminants, For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art. The terms “expression profile” or “gene expression profile” refers to any description or measurement of one or more of the genes that are expressed by a cell, tissue, or organism under or in response to a particular condition. Expression profiles can identify genes that are up-regulated, down-regulated, or unaffected under particular conditions. Gene expression can be detected at the nucleic acid level or at the protein level. The expression profiling at the nucleic acid level can be accomplished using any available technology to measure gene transcript levels. For example, the method could employ in situ hybridization, Northern hybridization or hybridization to a nucleic acid microarray, such as an oligonucleotide microarray, or a cDNA microarray. Alternatively, the method could employ reverse transcriptase-polymerase chain reaction (RT-TCR) such as fluorescent dye-based quantitative real time PCR (TaqMan® PCR). In the Examples section provided below, nucleic acid expression profiles were obtained using Affymetrix GeneChip® oligonucleotide microarrays, The expression profiling at the protein level can be accomplished using any available technology to measure protein levels, e.g., using peptide-specific capture agent arrays.

The terms “gene signature” and “signature genes” will be used interchangeably herein and mean the particular transcripts that have been found to be differentially expressed in some MASCs, as described herein. It is noted that differential levels of the corresponding proteins, will also be useful as a marker or signature for isolating and identifying MASCs, as described herein. The terms “gene”, “gene transcript”, and “transcript” are used somewhat interchangeable in the application. The term “gene”, also called a “structural gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. “Transcript” or “gene transcript” is a sequence of RNA produced by transcription of a particular gene. Thus, the expression of the gene can be measured via the transcript.

The invention also contemplates that the protein products of any of the genes in the MASCs gene signatures found for example in datasets and/or described in any of the Tables or Figures herein may have diagnostic value, as well as to serve as potential therapeutic targets.

### Kits

It is contemplated that all of the assays and MASC cells disclosed herein (e.g. components for determining the MASC expression profile) can be in kit form for use by a health care provider and/or a diagnostic laboratory.

In certain embodiments, the present disclosure provides for a kit comprising any of the MASC cells, or MASCs comprised within a biomatrix, applicable for research or therapeutic use.

In certain embodiments, the present disclosure provides for a kit comprising one or more probes and/or antibodies for detecting expression levels of one or more MASC markers as described herein.

Assays for the detection and quantitation of one or more of the MASC signature profiles can be incorporated into kits. Such kits may include probes for one or more of the genes from one or more signatures, as described herein, reagents for isolating and purifying nucleic acids from biological tissue or bodily fluid, reagents for performing assays on the isolated and purified nucleic acid, instructions for use, and reference values or the means for obtaining reference values in a control sample for the included genes.

### Molecular Biology

In accordance with the present invention, there may be numerous tools and techniques within the skill of the art, such as those commonly used in molecular immunology, cellular immunology, pharmacology, and microbiology. See, e.g., Sambrook et al. (2001) Molecular Cloning: A Laboratory Manual. 3rd ed. Cold Spring Harbor Laboratory Press: Cold Spring Harbor, N.Y.; Ausubel et al. eds. (2005) Current Protocols in Molecular Biology. John Wiley and Sons, Inc.: Hoboken, N.J.; Bonifacino et al. eds. (2005) Current Protocols in Cell Biology. John Wiley and Sons, Inc.: Hoboken, N.J.; Coligan et al. eds. (2005) Current Protocols in Immunology, John Wiley and Sons, Inc.: Hoboken, N.J.; Coico et al. eds. (2005) Current Protocols in Microbiology, John Wiley and Sons, Inc.: Hoboken, N.J.; Coligan et al. eds. (2005) Current Protocols in Protein Science, John Wiley and Sons, Inc.: Hoboken, N.J.; and Enna et al. eds. (2005) Current Protocols in Pharmacology, John Wiley and Sons, Inc.: Hoboken, N.J.

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

This invention will be better understood from the Experimental Details, which However, one skilled in the art will readily appreciate that the specific methods and results discussed are merely illustrative of the invention as described more filly in the claims that follow thereafter.

### EXAMPLES

### Example 1

Two mesenchymal stem cell types were compared—bone marrow derived MSCs (BM-MSCs) and placental-derived MSCs (PL-MSCs)—to the stem cells of the present invention, termed MASCs, or multipotent adult stem cells. According to the standards that characterize mesenchymal stem cells, it was determined, through comparative transcriptome analysis, that MASCs differ from MSCs. MASCs may be a better resource in regenerative research laboratories fora number of reasons. Additionally, MASC's may have therapeutic potential as described below for their ability to differentiate into many different tissue types.

RNA-seq is a recently developed approach to transcriptome profiling which provides more precise reads of transcriptomes than older methods. This approach is a type of Next Generation Sequencing (NGS) which is a term that applies to modern high-throughput sequencing technologies. Any high-throughput sequencing technology [9] can be used to perform RNA-seq. Here, Illumina MiSeq was used. All RNA-seqs were run in biological triplicate, as this allows the data to be considered legitimate according to the most recent standards for replicate number. The reads from RNA-seqs of BM-MSCs, PL-MSCs, and HF-derived MASCS (human foreskin-derived MASC's) were compared.

In this study, the transcriptomes of MASCs were characterized and the transcriptomic reads were compared with those of BM-MSCs and PL-MSCs in order to make deductions regarding the applicability of MASCs in the laboratories of the regenerative medicine field. To our knowledge, this was the first study to analyze the transcriptomic properties of MASCs, as these are a newly isolated line of cells. The focus, was on comparing the gene expression profile of MASCs to that of MSCs of different tissue derivatives, the MSCs being derived from bone marrow and placental origin, and the MASCs from HF cells. The gene expression profiles were Obtained from RNA-seqs and normalized before comparison through Microsoft Excel Spreadsheets.

### Methodology:

### Culture of HF-derived MASCs

Human foreskin-derived stem cells (HfSCs), passage number 20, were plated in 100 mm dishes at 100,000 cells per dish and cultured with Opti/Emem medium containing 5% pre-selected horse serum (HS-10). When the dishes reached 80% confluence, the cells were cultured in serum-free medium overnight in order to induce a growth-arrested state. 24 hours later, the RNA was isolated using the Trizol reagent protocol and the Qiagen RNEasy kit (Qiagen).

### Whole Transcriptome Shotgun Sequencing (RNAseq)

RNA-sect data for PL-MSCs and BM-MSCs were obtained from previous literature [10], and RNA-seq for the MASCs was at New York Medical College. All RNA-seqs were run with three biological replicates, and were performed by Illumina MiSeq.

Total RNA was extracted from cultured cells using the RNAeasy mini kit (Qiagen Sciences, Germantown, Md.). RNA concentration was determined by Qubit Fluorometric Quantitiation (Life Technologies, Carlsbad, Calif.). For each sample, 2 mcg of RNA was used to construct RNA-Seq cDNA libraries using the TruSeq RNA Sample Prep Kit v2 (Illumina, San Diego, Calif.), in accordance with the manufacturer's protocol using the poly-adenylated RNA isolation. The amplified cDNA fragments were analyzed using the 2100 Bioanalyzer (Agilent Technologies, Santa Clara, Calif.) to determine library quality and size. Sequencing of 75 bp paired-end reads was performed in the Illumina MiSeq.

### RNA-Seq Data Analysis

Raw sequence reads were de-multiplexed and trimmed for adapters by using the Illumina on-instrument MiSeq Reporter. Raw sequence reads downloaded from the Gene Expression Omnibus (GEO) website (http://www.ncbi.nlm.nih.gov/geo/) were trimmed using Trimmomatic (Bioinformaticsbtu170, 2014). Sequence reads of each sample were aligned to the reference hg19 genome Using TopHat; and quantified with the reference hg19 transcriptome using Cufflinks; differential expression of genes and transcripts in paired comparisons was obtained using Cuffdiff, in accordance with the Tuxedo pipeline (Nature Protocols 7:562, 2012). The mapped sequence reads were visualized under Intergative Genomics Viewer (IGV, Nature Biotechnology 29:24, 2011). The NIH Database for Annotation, Visualization, and Integrated Discovery (DAVID) was used to perform gene functional annotation clustering, with default options and annotation categories. The RNA-Seq data is available at the GEO website under accession GSE.

### Microsoft Excel Spreadsheet Data Analysis

Resulting from RNA-seqs were entire gene lists, which were entered into three master spreadsheets—one for each cell type. The reads from the RNA-seq analyses included the following information about each gene in the gene list: locus, gene ID, gene expression level in FPKM. From these master spreadsheets, further analysis of the data took place.

### DAVID

The functional annotation clustering of the gene list data was performed using NCBI's DAVID Bioinformatics Database. The clustering analysis was conducted using the highest stringency and the genes were matched to the Homo sapien species.

### Results:

Three of the most relevant gene lists were submitted into DAVID's functional annotation clustering system. These gene lists included: genes expressed in BM-MSCs exclusively, genes expressed in PL-MSCs exclusively, and genes expressed in HF-MASCs exclusively. As there were a total of 107 annotation clusters resulting from the three gene lists analyzed herein, only the annotation clusters that display the most apparent significance will be illustrated and discussed. The observations are separated between the three gene lists that were analyzed: PL-MSCs expressed only, BM-MSCs expressed only, and both BM-MSCs and PL-MSCs expressed only.

### PL-MSCs Expressed Only:

*The “Term” column provides highlighted gene families, protein domains, and important sites listed often in conjunction with the functional analysis accession number provided by DAVID's functional annotation cluster.

### BM-MSCs Expressed Only:

The most enriched annotation cluster (Annotation Cluster 1), with an enrichment score of 2.676, contained 3 genes that were involved in neurotrophin binding and receptor activity. These genes were NTRK3, NTRK1, and NTRK2, all members of the MAPK pathway.

TABLE 2
Important Annotation Clusters for the genes expressed only in BM-MSCs, comparatively.

| Annotation | Enrichment | | |
|---|---|---|---|
| Cluster | Score | Terms* | Genes Included |
| 1 | 2.676 | site:Interaction with PLC-gamma-1 | NTRK3, |
| | | site:Interaction with SHC1 | NTRK1, |
| | | IPR020777:Tyrosine-protein kinase, | NTRK2 |
| | | neurotrophic receptor | |
| | | GO:0043121~neurotrophin binding | |
| | | GO:0005030~neurotrophin receptor activity | |
| | | GO: 0042490~mechanoreceptor differentiation | |
| | | IPR002011:Tyrosine-protein kinase, receptor | |
| | | class II, conserved site | |

*The “Term” column provides highlighted gene families, protein domains, and important sites listed often in conjunction with the functional analysis accession number provided by DAVID's functional annotation cluster.

### BM-MSCs and PL-MSCs Expressed Only:

The most enriched annotation cluster of the BM and PL expressed genes, with an enrichment score of 3.0217, include 10 homeobox and homeodomain genes, and 2 homeobox-containing transcription factors DLX5 and EMX2. Annotation Cluster 2, with an enrichment score of 2.157 contain 13 of either homeodomain genes or homeobox genes.

TABLE 3
Important Annotation Clusters for the genes expressed only in BM-MSCs and PL-MSCs,
comparatively.

| Annotation | Enrichment | | |
|---|---|---|---|
| Cluster | Score | Terms* | Genes Included |
| 1 | 3.022 | IPR020479:Homeodomain, | HOXB3, HOXB4, HOXC8, |
| | | metazoa | HOXB2, HOXC9, HOXA3, |
| | | IPR0017970:Homeobox, | HOXB7, HOXA5, HOXC4, |
| | | conserved site | DLX5, HOXA6, EMX2 |
| | | SM00389:H0X | |
| 2 | 2.157 | DNA-binding | HOXB3, HOXB4, HOXC8, |
| | | region:Homeobox | HOXB2, HOXA3, HOXC9, |
| | | IPR001356:Homeodomain | HOXB7, HOXA5, DLX5, |
| | | Homeobox | HOXA6 HOXC4, EMX2, CERS4 |

### 144 Differentially Expressed Gene Panel Unique to MASCs

As shown below and in Table 10, we have identified a 144 gene panel of differentially expressed genes that are unique to the MASCs. These 144 genes are not expressed in human embryonic stem cells, human bone marrow- and placenta-derived mesenchymal stem cells, and human airway-derived differentiated smooth muscle cells.

### Genomics Data Outline

### (Glossary of Genes and Their Functions Located Below)

### Distinguishing Features of MASCs vs MSCs

The sets of differential genes that are expressed only by MASCs and NOT by MSCs fall into several categories:
Endodermal developmental genes (NKX6-1, ONECUT1)
Ectodermal, specifically neuroectodermal, developmental genes (ROBO2, RSPO2, SEMA6A, NETO1, FRMPD4, ZIC4, ZIC1, TMEFF2, CDH18, EPHA5, PCDH9)
This is consistent with the in vitro differentiation data for the MASCs→we have demonstrated that the MASCs can differentiate into all three dermal lineages, as opposed to MSCs which have been shown to be limited in their in vitro differentiation potential (mesodermal development).
Genes for cardiac development (TBX5)
Genes for germline development (LHX9, IGF2BP3, TBX4, MAB21L1, PAX3, FOXQ1, WNT3, SOX11, LRP4, PTCH1, TFAP2A).

Clusters of genes that are expressed by BOTH: MASCs and MSCs fall into other categories:
Mesodermal developmental pathways→this is consistent with well-reported in vitro differentiative potential of MSCs and consistent with the in vitro differentiation data for the MASCs.
(Some) Housekeeping and cell cycle genes→this is not surprising as these are genes that most cell types have in common
   EEF1A1→housekeeping genes robustly expressed by MASCs and NOT MSCs
   EEF1A2→housekeeping gene expressed by MASCs and NOT MSCs

These data illustrate that the MASCs as described herein, appear to have an unlimited proliferation potential. We hypothesize that this is in part due to the expression of EREG, since MASCs express EREG (epiregulin→necessary for sustained cell growth without senescence) and MSC's do not.

MASCs vs ESCs

Of the four critical ESC factors, MASCs do NOT express OCT4, SOX2, and Nanog; MASCs DO show expression of KLF-4.
MASCs express 126 of 130 pluripotency genes that ESCs express (pluripotency gene list compiled from several publications/reviews)
   This could contribute to the differentiative potential of the MASCs in comparison to the MSCs.
   Endodermal developmental genes that MASCs express which ESCs do NOT (based on MASC vs MSC comparison above):
   NKX6-1, ONECUT1
Ectodermal/Neuroectodermal developmental genes that MASCs express which ESCs do NOT (based on MASC vs MSC comparison above).
   RSPO2, FRMPD4, ZIC4, ZIC1, TMEFF2, CDH18, EPHA5, PCDH9
   Germline developmental genes that MASCs express which ESCs do not (based on MASC vs MSC comparison above):
   LUX9, TBX4, MAB21L1, PAX3, TFAP2A.
Cardiac developmental genes that MASCs express which ESCs do NOT (based on MASC vs MSC comparison above)
   TBXS
ESCs do Nar express EREG (epiregulin)→MSCs show NO expression of EREG either while MASCs do.
MASCs robustly express the housekeeping gene EEF1A1 and the ESCs do NOT express EEF1A1 (just as the MSCs do not either)→makes EEF1A1 a gene specific to MASCs as compared to the other three types of stem cells.

### MASCs vs Smooth Muscle Cells (A Differentiated Phenotype)

To date, 144 genes have been identified that are unique specifically to MASCs and are not expressed by BM-MSCs, PL-MSCs, ESCs, or smooth muscle cells
   This gene list includes SPINK6 and ROS1 (See Table 10)
EEF1A1 gene (housekeeping gene)→expressed by smooth muscle and MASCs but NOT MSCs OR ESCs
EEF1A2 gene (housekeeping gene)→expressed by MASCs but NOT by smooth muscle cells
EREG→NOT expressed by smooth muscle.

TABLE 4
GENES SOLELY EXPRESSED BY MASCs and NOT by BM-MSCs,
PL-MSCs, ESCs, OR Smooth Muscle Cells

| | |
|---|---|
| Endodermal Genes | ONECUT1 |
| Ectodermal/Neuroectodermal Genes | FRMPD4, ZIC4, ZIC1, TMEFF2, |
| | CDH18, EPHA5, PCDH9 |
| Genes Related to Germline Development | LHX9, PAX3 |
| Others | EREG (epiregulin) |

### Glossary of Genes and Functions:

Endodermal Genes:

ONECUT1: Gene encodes for proteins enriched in, the liver, where it stimulates development

NKX6-01: Gene is required for the development of pancreatic beta cells

### Ectodermal/Neuroectodermal Genes:

ROBO2: Cellular migration during neuronal and cerebral cortex development

RSPO2: Regulates craniofacial patterning and morphogenesis

SEMA6A: Expressed in developing neural tissue

NETO1: Involved in the development and maintenance of neuronal circuitry

FRMPD4: Positive regulator of dendritic spine morphogenesis and density

ZIC4: Involved in the development of the cerebellum

ZIC1: Involved in neurogenesis

TMEFF2: Involved in the development of hippocampal and mesencephalic neurons

CDH18: Expressed specifically in the central nervous system for development

EPHA5: Development of olfactory neurons in embryonic olfactory pathway

PCDH9: Codes for proteins involved in cell adhesion in neural tissues in the presence of calcium and also encodes proteins involved in signaling at neuronal synaptic junctions

### Cardiac Genes:

IBM: Gene for heart development and cardiac progenitor differentiation

### Germline Development Genes:

LHX9: involved in gonadal development→potential germline stem cell marker (literature)

IGF2BP3: Strongly expressed during embryonic development→also a strong candidate for a germline stem cell marker (literature)

TBX4: Gene encodes transcription factors involved in the regulation of embryonic developmental processes

MAB21L1: Early gene in embryogenesis→8 h post-fertilization in chicks. *Highly conserved across phyla

PAX3: Involved in dematomyotome early development

FOXQ1: Involved in embryonic development cell cycle regulation, tissue-specific gene expression, cell signaling, and tumorigenesis. Plays a role in hair follicle differentiation,

TFAP2A; Expressed in early neural crest cells migrating from cranial fouls during the closure of the neural tube. Plays a role in craniofacial morphogenesis.

PTCH1: Receptor for Sonic Hedgehog. Plays a role in formation of embryonic structures and is a tumor suppressor.

LRP4: Plays a key role in formation and maintenance of the neuro-muscular junction

WNT3: Regulates cell fate and patterning during embryogenesis. Plays a role in early development of the neural tube.

SOX11: Regulation of embryonic development and cell fate

### Other:

EREG (epiregulin): Necessary for sustained cell growth without senescence

EEF1A1: Elongation factor→delivers tRNA to the ribosome. Expressed in brain, placenta, lung, kidney, and pancreas.

EEF1A2: Elongation factor deliver tRNA to the ribsome. Expressed in brain, heart, and skeletal muscle.

### SUMMARY

We were successful in further characterizing our MASCs through the obtaining of RNA-seq reads that were annotated and analyzed. We provided a greater understanding of specific. characteristics of MASCs. Their lack of expression of Nanog and OCT-4 was confirmed by the observation of no expression in MASCs of the homeodomain genes that would make these genes, and their inability to form teratomas in vivo was confirmed by their lack of the TNF-receptor family proteins which mediate the recruitment of anti-apoptotic proteins. This correlation between the newly derived data and earlier information about MASCs indicated that the present tests were accurate.

The 144 gene panel of differentially expressed genes in MASCs reflects advantages of MASCs over MSC's including both BM- and PL-derived MSCs as well as embryonic stem cells and differentiated smooth muscle cells. This differential method of characterizing a population of MASC's can be utilized to Additional experiments will assess the efficacy of the MASCs and benefits of these cell types in vitro, and eventually in vivo. Efficiency and applicability of the cells in a laboratory setting can more easily and visually be assessed when using live samples, rather than observing the differences in gene expression within entire transcriptomes of three different cells.

### Gene IDs

TABLE 5
Gene IDs for differentially Expressed markers expressed by MASCs

| | |
|---|---|
| Gene | Gene ID |

Gene IDs for genes on the MASC vs MSC markers list

| | |
|---|---|
| EEF1A1 | NCBI Reference Sequence: NP_001393 |
| EREG | NCBI Reference Sequence: NM_001432 |
| COL1A1 | NCBI Reference Sequence: NG_007400 |
| LHX9 | NCBI Reference Sequence: XM_005245350 |
| SEMA3D | NCBI Reference Sequence: NG_051329 |

Endodermal Genes-MASCs express but MSCs do not

| | |
|---|---|
| NKX6-1 | NCBI Reference Sequence: NM_006168 |
| ONECUT1 | NCBI Reference Sequence: NM_004498 |

Ectodermal/Neuroectodermal Genes MASCs express but MSCs do not

| | |
|---|---|
| ROBO2 | NCBI Reference Sequence: NG_027734 |
| RSPO2 | NCBI Reference Sequence: NM_178565 |
| SEMA6A | NCBI Reference Sequence: NM_001300780 |
| NETO1 | NCBI Reference Sequence: NM_138966 |
| FRMPD4 | NCBI Reference Sequence: NG_016419 |
| ZIC4 | NCBI Reference Sequence: NG_009242 |
| ZIC1 | NCBI Reference Sequence: NG_015886 |
| TMEFF2 | NCBI Reference Sequence: NM_016192 |
| CDH18 | NCBI Reference Sequence: NM_004934 |
| EPHA5 | NCBI Reference Sequence: NM_004439 |
| PCDH9 | NCBI Reference Sequence: NG_011876 |

Cardiac Development Marker Genes-MASCs express but MSCs do not

| | |
|---|---|
| TBX5 | NCBI Reference Sequence: NG_007373 |

Germline Development Marker Genes-MASCs express but MSCs do not

| | |
|---|---|
| LHX9 | NCBI Reference Sequence: NM_005245350 |
| IGF2BP3 | NCBI Reference Sequence: NM_006547 |
| TBX4 | NCBI Reference Sequence: NG_008080 |
| MAB21L1 | NCBI Reference Sequence: NG_016811 |
| PAX3 | NCBI Reference Sequence: NG_011632 |
| FOXQ1 | NCBI Reference Sequence: NM_033260 |
| WNT3 | NCBI Reference Sequence: NG_008084 |
| SOX11 | NCBI Reference Sequence: NG_050751 |
| LRP4 | NCBI Reference Sequence: NG_021394 |
| PTCH1 | NCBI Reference Sequence: NG_007664 |
| TFAP2A | NCBI Reference Sequence: NG_016151 |

Housekeeping/Cell Cycle Marker Genes-MASCs express but MSCs do not

| | |
|---|---|
| EEF1A1 | NCBI Reference Sequence: NP_001393 |
| EEF1A2 | NCBI Reference Sequence: NG_034083 |

TABLE 6
Gene IDs for markers expressed by MSCs (BMs and PLs)
and NOT by MASCs

| Gene | Gene ID |
|---|---|
| Desmin | NCBI Reference Sequence: XM_006265857 |
| RNASE1 | NCBI Reference Sequence: NM_198235 |
| GPC4 | NCBI Reference Sequence: NG_012498 |
| ISLR | NCBI Reference Sequence: NM_005545 |
| FGF-7 | NCBI Reference Sequence: NG_029159 |
| CD4 | NCBI Reference Sequence: NG_027688 |
| Cd120b | NCBI Reference Sequence: NG_029791 |

TABLE 7
Gene IDs for markers expressed by MASCs and NOT by ESCs

| | |
|---|---|
| Gene | Gene ID |

Endodermal Genes

| | |
|---|---|
| NKX6-1 | NCBI Reference Sequence: NM_006168 |
| ONECUT-1 | NCBI Reference Sequence: NM_004498 |

Ectodermal/Neuroectodermal Genes

| | |
|---|---|
| RSPO2 | NCBI Reference Sequence: NM_178565 |
| FRMPD4 | NCBI Reference Sequence: NG_016419 |
| ZIC4 | NCBI Reference Sequence: NG_009242 |
| ZIC1 | NCBI Reference Sequence: NG_015886 |
| TMEFF2 | NCBI Reference Sequence: NM_016192 |
| CDH18 | NCBI Reference Sequence: NM_004934 |
| EPHA5 | NCBI Reference Sequence: NM_004439 |
| PCDH9 | NCBI Reference Sequence: NG_011876 |

Germline Development Marker Genes

| | |
|---|---|
| LHX9 | NCBI Reference Sequence: XM_005245350 |
| TBX4 | NCBI Reference Sequence: NG_008080 |
| MAB21L1 | NCBI Reference Sequence: NG_016811 |
| PAX3 | NCBI Reference Sequence: NG_011632 |
| TFAP2A | NCBI Reference Sequence: NG_016151 |

Cardiac Development Marker Genes

| | |
|---|---|
| TBX5 | NCBI Reference Sequence: NG_007373 |

Other Genes

| | |
|---|---|
| EREG | NCBI Reference Sequence: NM_001432 |
| EFF1A1 | NCBI Reference Sequence: NP_001393 |

TABLE 8
Gene IDs for markers expressed by ESCs and NOT
by MASCs → 3 of the 4 critical factors

| Gene | Gene ID |
|---|---|
| Oct4 | NCBI Reference Sequence: NM_112957 |
| Sox2 | NCBI Reference Sequence: NG_009080 |
| Nanog | NCBI Reference Sequence: NM_024865 |

TABLE 9
Gene IDs for markers expressed by MASCs and NOT
by Smooth Muscle Cells (SMCs)

| Gene | Gene ID |
|---|---|
| EREG | NCBI Reference Sequence: NM_001432 |
| EEF1A2 | NCBI Reference Sequence: NG_034083 |

TABLE 10
Gene IDs for markers expressed SOLELY by MASCs and NOT by
BM-MSCs, PL-MSCs, ESCs, OR Smooth Muscle Cells

| | |
|---|---|
| Gene | Gene ID |

Endodermal Genes

| | |
|---|---|
| ONECUT1 | NCBI Reference Sequence: NM_004498 |

Ectodermal/Neuroectodermal Genes

| | |
|---|---|
| FRMPD4 | NCBI Reference Sequence: NG_016419 |
| ZIC4 | NCBI Reference Sequence: NG_009242 |
| ZIC1 | NCBI Reference Sequence: NG_015886 |
| TMEFF2 | NCBI Reference Sequence: NM_016192 |
| CDH18 | NCBI Reference Sequence: NM_004934 |
| EPHA5 | NCBI Reference Sequence: NM_004439 |
| PCDH9 | NCBI Reference Sequence: NG_011876 |

Germline Development Marker Genes

| | |
|---|---|
| LHX9 | NCBI Reference Sequence: XM_005245350 |
| PAX3 | NCBI Reference Sequence: NG_011632 |

Other Genes

| | |
|---|---|
| EREG | NCBI Reference Sequence: NM_001432 |
| SPINK6 | NCBI Reference Sequence: NM_205841 |
| ROS1 | NCBI Reference Sequence: NG_033929 |
| SNORD79 | NCBI Reference Sequence: NR_003939 |
| MIR3074 | NCBI Reference Sequence: NG_027833 |
| SNORD35B | NCBI Reference Sequence: NR_001285 |
| SNORD58C | NCBI Reference Sequence: NR_003701 |
| SNORD11 | NCBI Reference Sequence: NR_003031 |
| MIR630 | NCBI Reference Sequence: NR_030359 |
| SNORD116-23 | NCBI Reference Sequence: NR_003337 |
| SNHG25 | NCBI Reference Sequence: NR_132278 |
| MIR6826 | NCBI Reference Sequence: NR_106884 |
| SNORD116-26 | NCBI Reference Sequence: NR_003340 |
| SNORD127 | NCBI Reference Sequence: NR_003691 |
| MIR218-1 | NCBI Reference Sequence: NG_047105 |
| SNORA77 | NCBI Reference Sequence: NG_029589 |
| SNORA26 | NCBI Reference Sequence: NR_003016 |
| SNORD94 | NCBI Reference Sequence: NR_004378 |
| SNORA80B | NCBI Reference Sequence: NG_012105 |
| MKX-AS1 | NCBI Reference Sequence: NR_121652 |
| HOXC-AS3 | NCBI Reference Sequence: NR_047506 |
| ZNF560 | NCBI Reference Sequence: NG_054924 |
| SNHG24 | NCBI Reference Sequence: NG_045000 |
| KIAA1024L | NCBI Reference Sequence: NM_001257308 |
| XXLYT1-AS2 | NCBI Reference Sequence: NM_001084309 |
| LOC101926940 | NCBI Reference Sequence: NC_000005 |
| HOTTIP | NCBI Reference Sequence: NR_037843 |
| KCCAT198 | NCBI Reference Sequence: NR_131986 |
| MIR137HG | NCBI Reference Sequence: NR_046105 |
| C1GALT1C1L | NCBI Reference Sequence: NM_001101330 |
| TREML3P | NCBI Reference Sequence: NR_027256 |
| LINC01291 | NCBI Reference Sequence: NR_125792 |
| LOC100132735 | NCBI Reference Sequence: NC_000006 |
| ZNF826P | NCBI Reference Sequence: NR_036455 |
| SERPINB10 | NCBI Reference Sequence: NM_005024 |
| LINC00333 | NCBI Reference Sequence: NR_046871 |

### REFERENCES:

1. stemcells.nih.gov/info/basics/pages/basics1.aspx

2. Dominici M. et al. Minimal criteria for defining multipotent mesenchymal stromal cells. The International Society for Cellular Therapy position statement. Cytotherapy 8, 315-317 (2006).

3. Bianco P., Robey, P. G. & Simmons P. J. Mesenchymal stem cells: revisiting history, concepts, and assays. Cell Stem Cell 2, 313-319 (2008).

4. Wagner W. et al. Aging and Replicative Senescence Have Related Effects on Human Stem and Progenitor Cells. PLoS ONE doi: (2009).10.1371/journal.pone.0005846

5. Trivedi P. & Hernatti P. Derivation and immunological characterization of mesenchymal stromal cells from human embryonic stem cells. Exp. Hematol. 36, 350-359 (2008).

6. Pittenge M F, Mackay A M, Beck S C, Rama K. Jaiswal R K, Douglas R, Mosca J D, Moorman M A, Simonetti D W, Craig S, Marshak D R. Multilineage Potential of Adult Human Mesenchymal Stem Cells. Science 28, 143-147, 1999.

7. Kern S., Eichler H., Stoeve J., Klüter H. & Bieback K. Comparative Analysis of Mesenchymal Stem Cells from Bone Marrow, Umbilical Cord Blood, or Adipose Tissue. STEM CELLS 24, 1294-1301 (2006).

8. Davies O. G., Cooper P. R., Shelton R. M., Smith A. J. & Scheven B. A. A comparison of the in vitro mineralisation and dentinogenic potential of mesenchymal stem cells derived from adipose tissue, bone marrow and dental pulp. J. Bone Miner. Metab. 33, 371-382 (2014).

8a. in 't Anker, P S, Scherjon, S A, van der Keur, K C, de Groot-Swings G M J S, Claas F F J, Fibbe W E, Kanhaia H H H. Isolation of Mesenchymal Stem Cells of Fetal or Maternal Origin from Human Placenta. Stem Cells 22, 1338-1345, 2004

9. Holt B A, Jones S. J. The new paradigm of flow cell sequencing. Genome Res. 2008;18:839-846.

10. B. Roson-Burgo, P. Sanchez-Guijo, C. D. Cañizo, J. D. L. Rivas. Transcriptomic portrait of human mesenchymal stromal/stem cells isolated from bone marrow and placenta. BMC Genomics, 15 (2014), p. 910 dx.doi.org/10.1186/1471-2164-15-910

11. Fu W., Li J., Chen G., Li Q., Tang X., Zhang C. Mesenchymal stem cells derived from peripheral blood retain their pluripotency, but undergo senescence during long-term culture. Tissue Engineering Part C: Methods: 2015; 21(10):1088-1097. doi: 10.1089/ten.tec.2014.0595.

12. Hare, I., Gencheva, M., Evans, R., Fortney, J., Piktel, D., Vos, J. A., . . . Gibson, L. F. (2016). In Vitro Expansion of Bone Marrow Derived Mesenchymal Stem Cells Alters DNA Double Strand Break Repair of Etoposide Induced DNA Damage. Stem Cells International, 2016, 8270464. doi.org/10.1155/2016/8270464

13. Grotendorst G R, Okochi H, Hayashi N. A novel transforming growth factor beta response element controls the expression of the connective tissue growth factor gene. Cell Growth Differ. 1996;7:469-480.

14. Morrison, D. K, (2012). MAP kinase pathways. Cold Spring Harb. Perspect. Biol. 4, a011254.

15. Murry, C. E., & Keller, G. (2008). Differentiation of embryonic stem cells to clinically relevant populations: lessons from embryonic development. Cell, 132(4), 661-680.

### Example 2

Human MASC vs Human BM and PL-MSC RNA-seq comparison, and Human MASC vs Human ESC RNA-seq Comparison

### Genomic Data:

### MASC vs BM and PL-MSCs

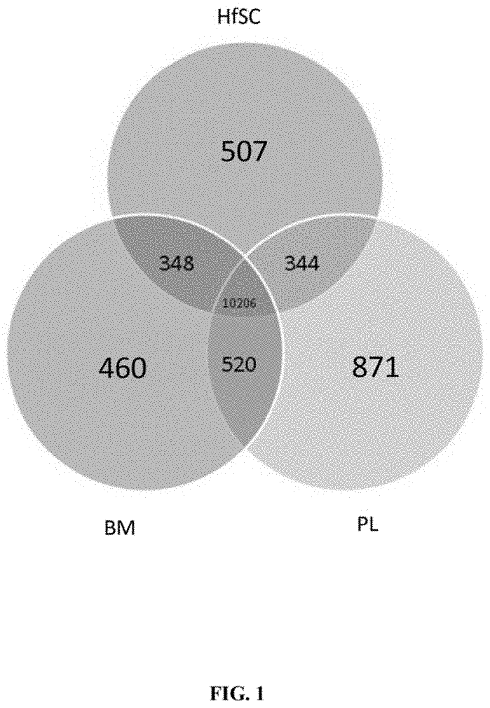

589 genes were identified to be expressed only by MASCs and by neither BM-MSCs nor PL-MSCs. 511 genes were identified to be expressed only by BM-MSCs and by neither MASCs nor PL-MSCs. 388 genes were identified to be expressed only by PL-MSC: and by neither MASCs nor BM-MSCs. The threshold for designating a significant expression level was set to greater than 1 fragment per kilobase per million (>1 FPKM). 10,609 genes were found to be significand expressed by both MASCs and BM-MSCs but not PL-MSCs. 10,732 genes were found to be significantly expressed by both MASCs and PL-MSCs but not BM-MSCs. 11,034 genes were found to be significantly expressed by both BM-MSCs and PL-MSCs but not MASCs. See FIG. 1.

Functional clustering annotation and pathway analysis conducted using the Database for Annotation, Visualization, and Integrated Discovery (DAVID) was then performed on each of these gene sets. The KEGG database in DAVID was used specifically for pathway analysis.

The clustering analysis for the genes expressed only by MASCs and by neither BM-MSCs nor PL-MSCs was performed using an enrichment score threshold for significance of clustering of greater than or equal to 1.3, The following clusters were found to be significant in this gene set: neurogenesis, embryonic epithelial tube formation, early embryogenesis, vasculature development, cell migration, mitotic activity, and meiotic activity.

Gene lists submitted into DAVID's functional annotation clustering system included: genes expressed in BM-MSCs exclusively, genes expressed in PL-MSCs exclusively, and genes expressed in MASCs exclusively.

### PL-MSCs Expressed Only:

The findings in Annotation Cluster 8 of the PL-MSC-expressed gene list found in Table 1 suggest a major source fix the proliferation, differentiation, and transformation capacity of PL-MSCs. The genes listed: PTPRB, PTPRE, PTPRH, and PTPRN are all part of the protein-tyrosine phosphatase family, which provide a link in the MARK pathway. The genes included in Annotation Cluster 12, as displayed in Table 1 are members of the TGF-beta superfamily of proteins.

### BM-MSCs Expressed Only:

The most enriched annotation cluster (Annotation Cluster 1), with an enrichment score of 2.676, contained 3 genes that were involved in neurotrophin binding and receptor activity. These genes were NTRK3, NTRK1, and NTRK2, all members of the MAPK pathway.

### BM-MSCs and IPL-MSCs Expressed Only:

The most enriched annotation cluster of the BM and PL expressed genes, with an enrichment score of 3.0217, includes 10 homeobox and homeodomain genes, and 2 homeobox-containing transcription factors DLX5 and EMX2. Annotation Cluster 2, with an enrichment score of 2.157 contains 13 of either homeodomain genes or homeobox genes.

### MASC vs ESC

The results showed that ESCs significantly express 2,440 genes that the MASCs do not express according to our threshold. Among these genes unique in expression to ESCs are the well-known pluripotency genes NANOG and SOX2. Using the threshold for genetic expression of 1.00, the Excel sort showed 1,014 genes that MASCs significantly expressed and the ESCs did not express. Of the 1,014 genes uniquely significantly expressed by MASCs, 23 were discovered to be small nucleolar RNA (SNORAs), and for most of the SNORAs, very little are known about the individual genes and the function of these genes remains to be defined. In addition, there were 11 SNORDs, which are small nucleolar RNAs that are non-coding, and to likewise, insufficient information is known about these SNORDs regarding their function and role in an organism. There were also 22 microRNAs expressed by the MASCs. MicroRNAs are very short (roughly 22 nucleotides), single-stranded, non-coding RNAs which regulate gene expression post-transcriptionally. It's believed that the function of miRNAs include cell differentiation and the protection of cell identity.

Of the 1,014 genes only expressed by MASCs, some genes were determined to be of interest and worth elaborating in detail in. These genes are SPINK6 and ROS1. SPINK6 is a serine peptidase inhibitor, Kazal Type 6. This gene is protein-coding (Gene Cards: www.genecards.org/cgi-bin/carddisp.pl?gene=SPINK6). SPINK6 was reported to inhibit KLk5, KLK4, and KLK14 in a 2011 scientific study (www.ncbi.nlm.nih.gov/pubmed?cmd=search&term=21439340&dopt=b ROS1 is a proto-oncogene and a receptor tyrosine kinase. ROS1 has been linked to gastric cancer, ovarian cancer, colorectal cancer, and angiosarcoma among others. (civic.genome.wustl.edu/sources/866/summary)

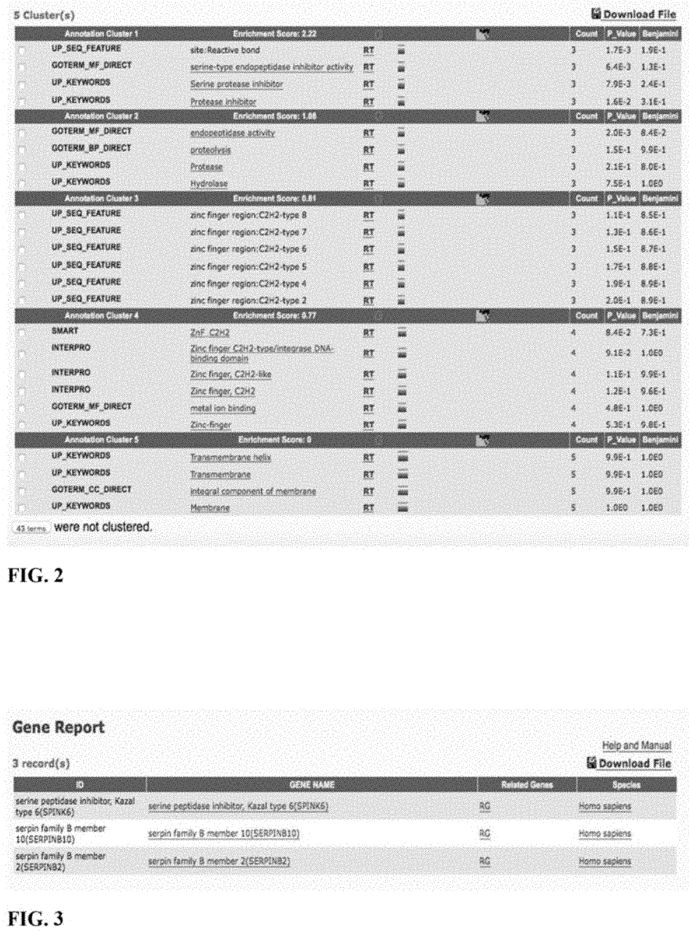

The table in FIG. 2 shows the functional annotation clustering results Of the DAVID software using the gene list of 1,014 genes found to be only expressed in HA when compared with ESCs. DAVID found five annotation clusters. Threshold for enrichment scores was set to be 1.3, so only Annotation Cluster 1 was deemed of significance.

FIG. 3 shows the gene, report for Functional Annotation Cluster I, showing that SPINK6, SERPINB10, and SERPINB2 are within the cluster.

### Results of DAVID Using 2,440 ESC Unique Gene List

The functional annotation cluster analysis showed 13 clusters with enrichment scores above 1.3. Again, the threshold for enrichment scores is set to be 1.3. Of these clusters, Functional Annotation Cluster #4 contained genes of interest. Cluster #4 had an enrichment score of 2.27. Cluster #4 contained the genes BARX1, HMX2, LHX1, LHX5, POU2F3, POU3F1, VENTX, GSC, IRX1, MNX1, ONECUT1, OTX2, and PHOX2A. Most of these genes play a role in organ or tissue development. For example, BARX1 plays a functional role in craniofacial development and tooth development (science.sciencemag.org/content/282/5391/1136)

HMX2 is a transcription factor that plays a role in hypothalamus and inner ear development. (www.uniprot.org/uniprot/A2RU54#function) LHX1 plays a role in renal and urogenital development. (www.ncbi.nlm.nih.gov/gene?cmd=Retrieve&dopt=full_report&list_uids=3975) POU3F1 may play a role in early stages of embryogenesis and neurogenesis. (www.uniprot.org/uniprot/Q03052), MNX1 plays a role in pancreatic development. (www.uniprot.org/uniprot/P50219#function). OTX2's encoded protein is involved in the development of craniofacial, sensory organ, and brain. (www.ncbi.nlm.nih.gov/gene?cmd=Retrieve&dopt=full_report&list_uids=5015) PHOX2A has a vital function in the autonomic nervous system development.

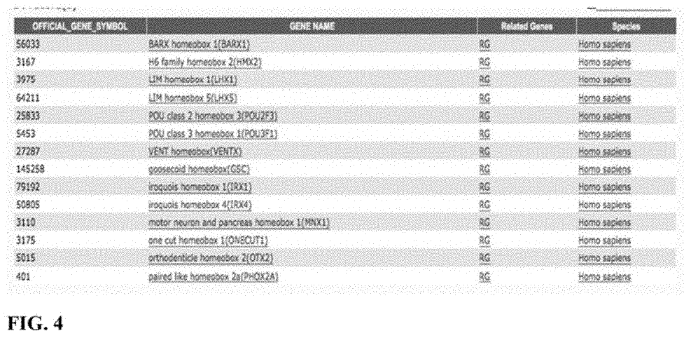

See FIG. 4 for DAVID functional annotation clustering gene report for Cluster 44.

### Results of Proposed Pluripotency Gene Comparison Between ESC and Hfs

The gene expression levels of these proposed pluripotency genes between ESCs and HFs were compared. The MASCs express 126 out of the 130 pluripotency genes analyzed, which is very significant as it shows that MASCs are quite similar to ESCs in regards to proposed pluripotency genes.

From this analysis, only four genes were found to be expressed by ESCs but not MASCs. Again, the threshold for expression is maintained at 1.00 FPXM. These four genes are POU5F1, UTF1, KCNAB3, and MDFI.

POU5F1, also known as OCT-4 (Octamer-binding transcription factor 4), is widely known as a key pluripotency transcription factor. When OCT-4 is overexpressed, the mesendoderm differentiates (Niwa, H., Miyazaki, J. & Smith, A. (Quantitative expression of Oct-3/4 defines differentiation, dedifferentiation or self-renewal of ES cells. Nat Genet. 24, 372-376 (2000).) UTF1, undifferentiated embryonic cell transcription factor 1, can function as a transcriptional repressor and is essential for embryonic carcinoma and ESCs to differentiate. It's linked with chromatin (www.ncbi.nlm.nih.gov/gene?cmd=Retrieve&dopt=full_report&list_uids=8433). KCNAB3 encodes a protein which is part of the beta subunits and belongs to the “potassium channel, voltage gated, shaker-related subfamily (www.ncbi.nlm.nih.gov/gene?cmd=Retrieve&dopt=full_report&list_uids=9196). Lastly, MDFI is a transcription factor which represses myogenesis. The axin regulation of WNT and INK pathways is also influenced by MDFI. (www.uniprot.org/uniprot/Q997504function)

### MASC vs Smooth Muscle Cells

Data analysis has shown that there are 668 genes that the human MASCs express which the human smooth muscle cells do not. In addition, there are 1,164 genes that are expressed by human smooth muscle cells that are not expressed by the human MASCs. A number of genes expressed solely by human MASCs and not by human BM-MSCs, human PL-MSCs, human ESCs, and human smooth muscle cells have been identified. See Table 10.

### Summary

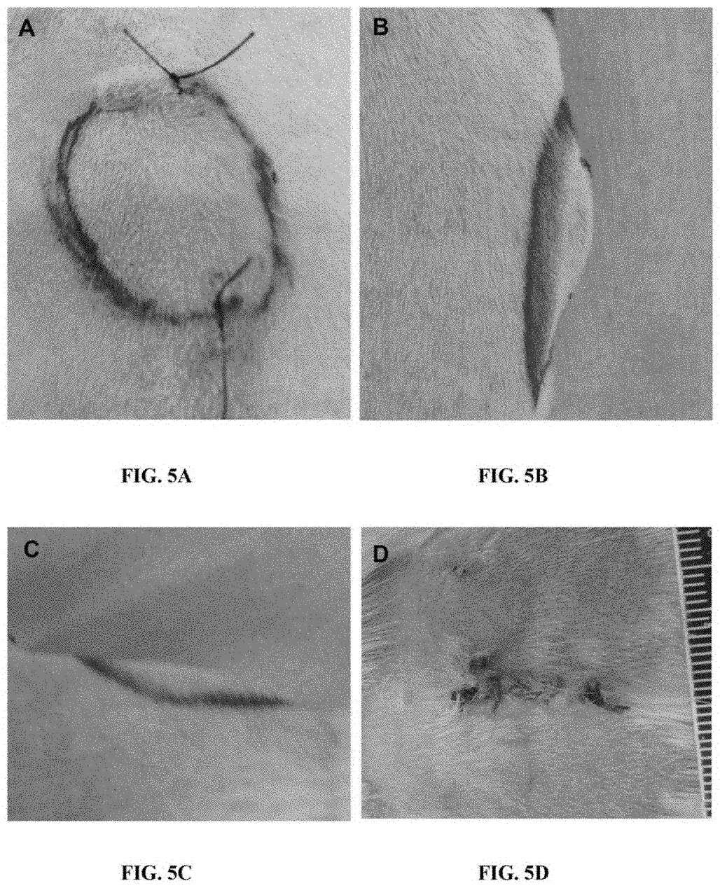

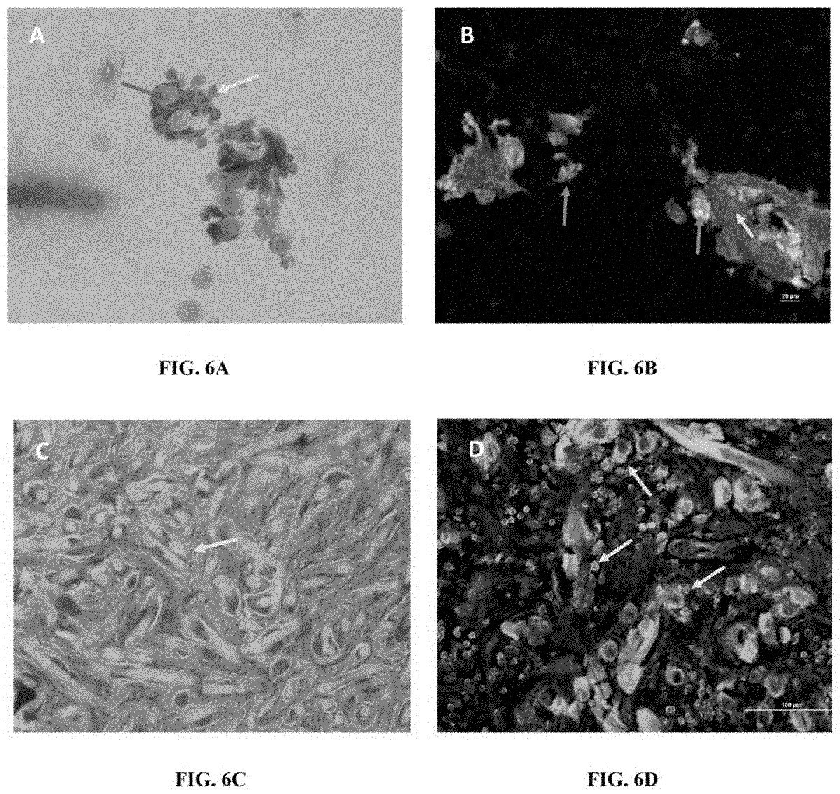

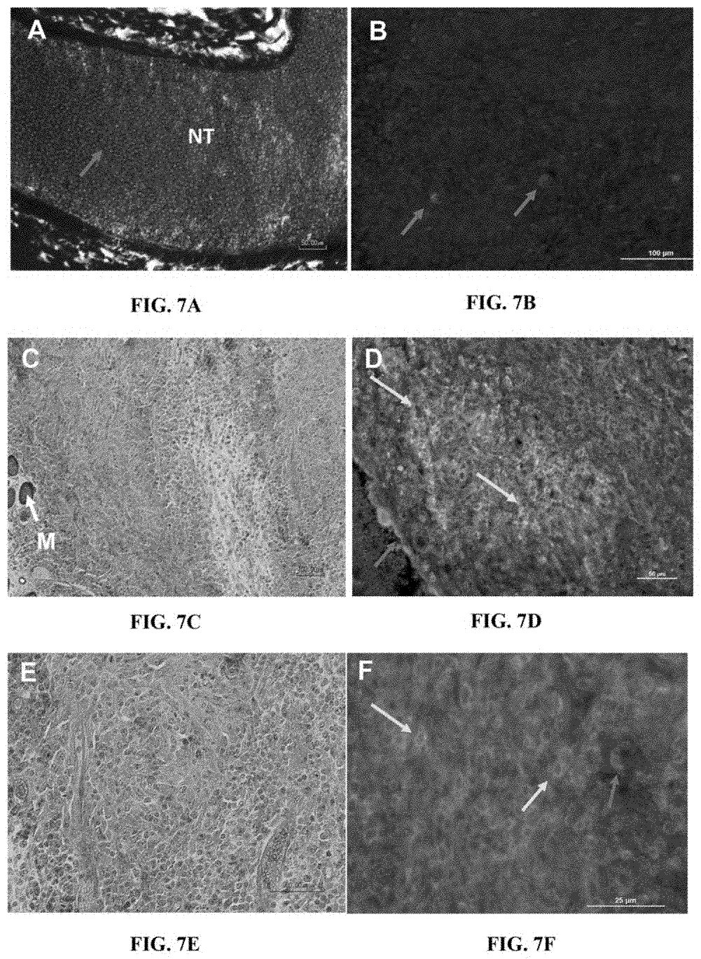

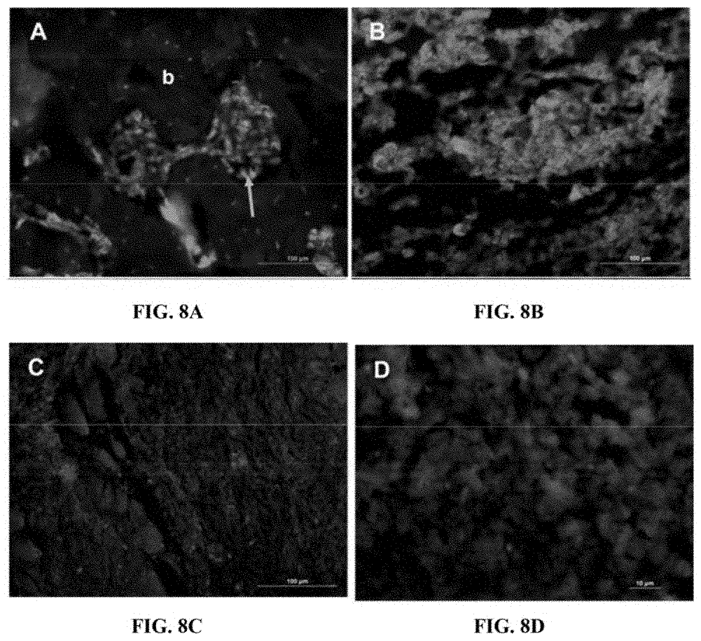

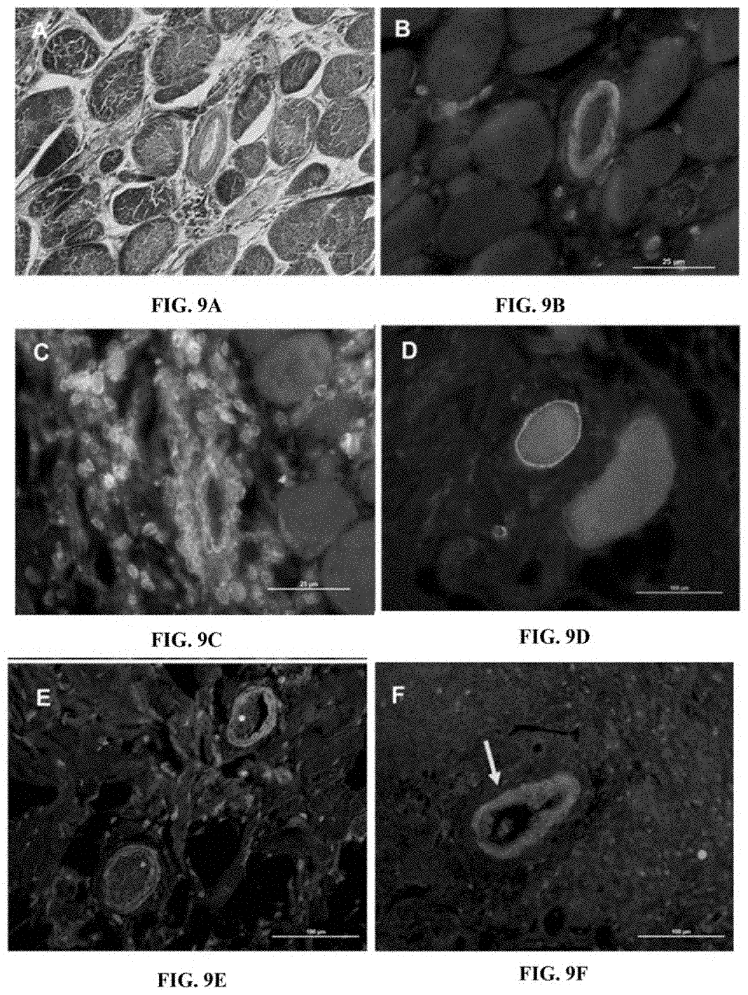

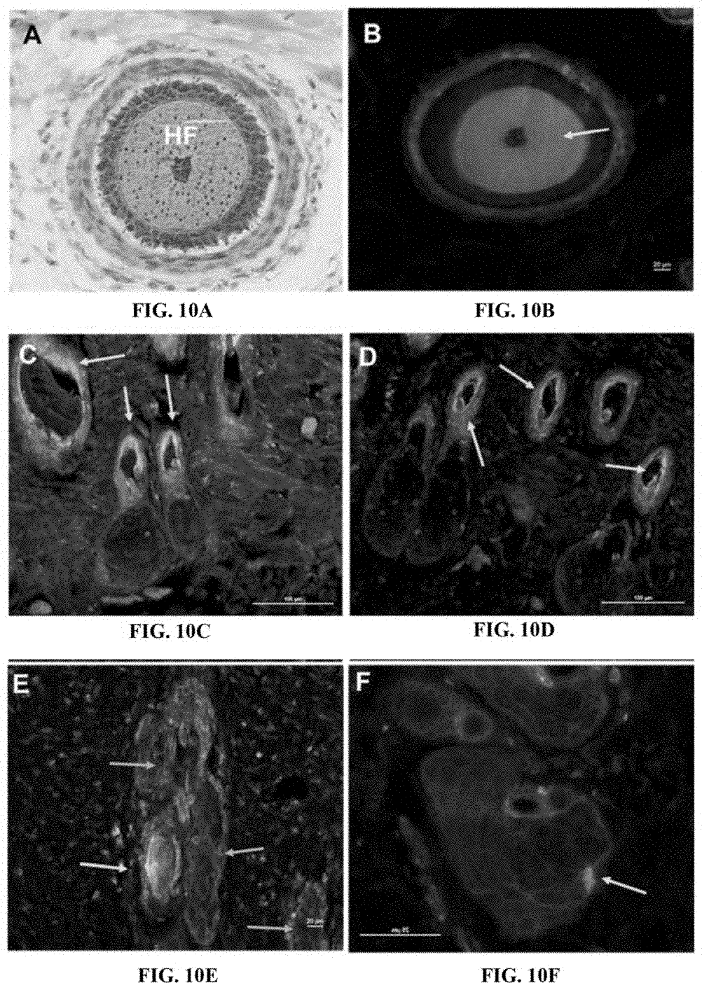

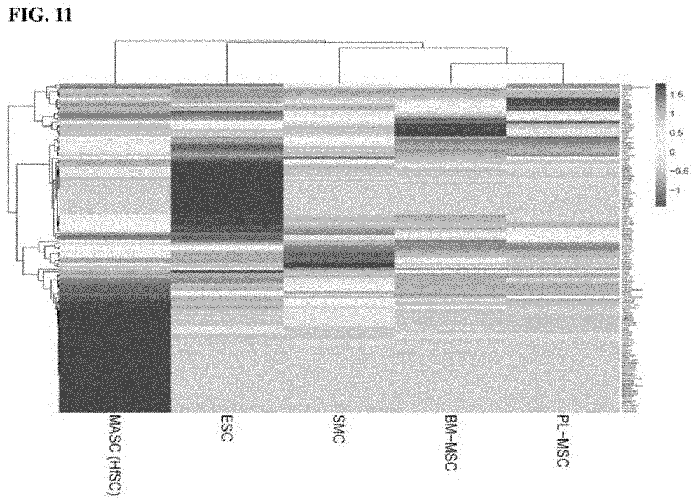

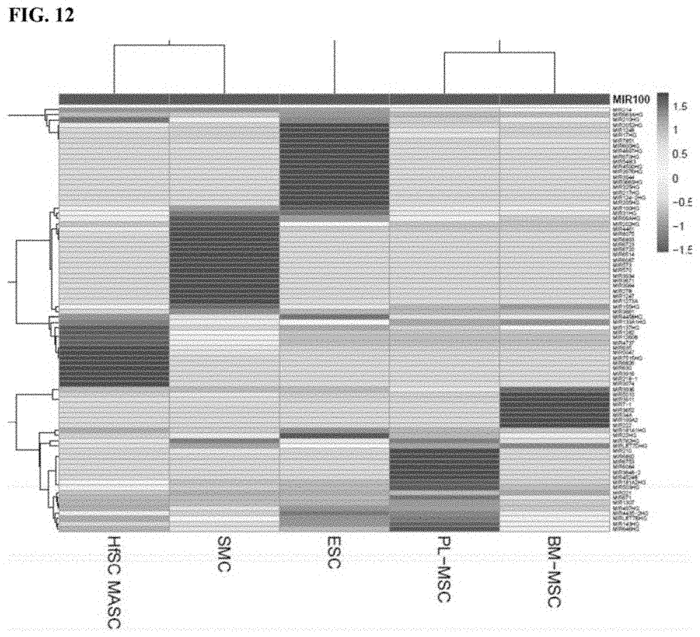

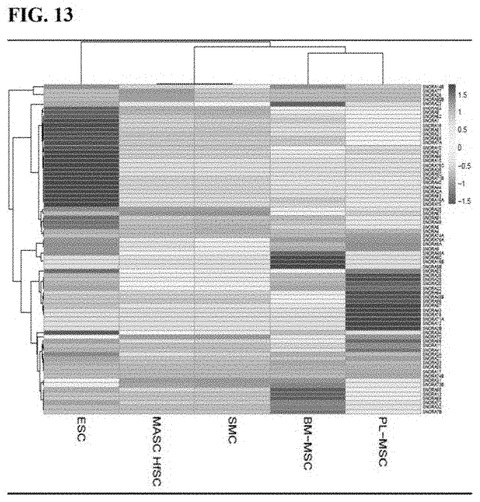

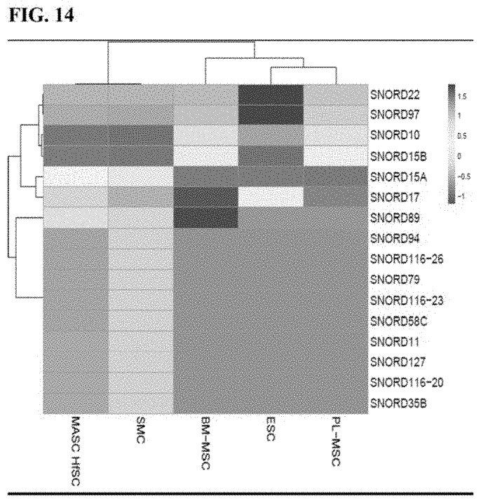

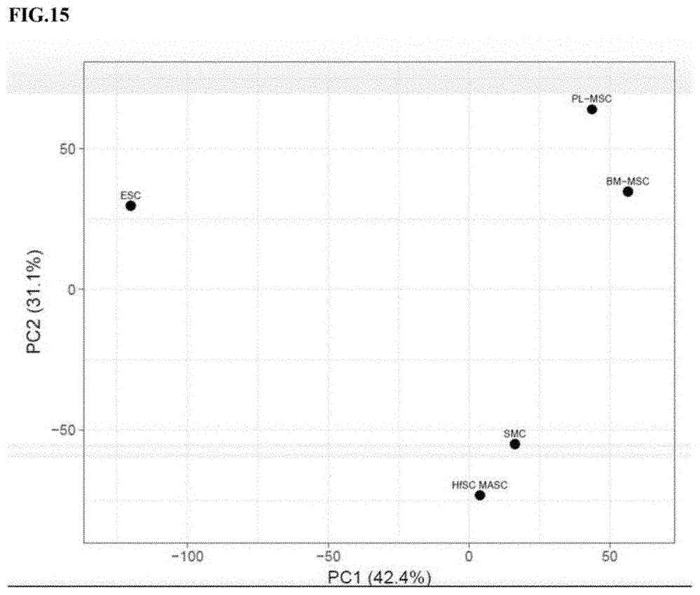

It was previously demonstrated that MASCS isolated from rats could differentiate into phenotypes of all 3 primary germ layers and human MASCs could differentiate into phenotypes from both the mesodermal and ectodermal lineages. MSCs have been shown to be limited to differentiating into phenotypes of the mesodermal lineage with little evidence of ectodermal differentiation. To date, we have demonstrated that human MASCs isolated from foreskin are not only able to differentiate into phenotypes from the mesodermal and ectodermal lineages but they are also able to differentiate into phenotypes from the endodermal lineage, making them cells that are capable of differentiating into phenotypes of all three primary germ layers. After establishing the extent of their differentiative potential in vitro, we then sought out to investigate their ability to differentiate in vivo. Previously published data has shown that MSCs do not tend to differentiate in vivo as the cells either die or go quiescent. A xenogenic study using human MASCs in rat was performed. This study demonstrated that human MASCs differentiate in vivo. It also showed that the human MASCs failed to illicit an immune response in rats. These results are shown in the images and FIGS. 5-10. The mechanism for this lack of immune response remains unknown. After demonstrating their ability to differentiate in vivo, the next step was to characterize these cells based on their whole transcriptome using whole genome RNA-sequencing. RNA-seq was performed on human MASCs, specifically human foreskin-derived MASCs (HfSCs). These HfSCs are the cells that have been used in each of the experiments mentioned above. The RNA-seq of the HfSCs was then compared to RNA-seq databases from human bone marrow-derived MSCs (BM-MSCs), human placenta-derived MSCs (PL-MSCs), human embryonic stem cells (ESC), and human smooth muscle cells (SMCs). The latter comparison was performed in order to observe differences in the transcriptomes of stem cells versus a differentiated cell. These comparisons of different RNA-seq have been and are currently being used to identify markers specific to human MASCs in an attempt to understand the reason for the difference in their behavior from other stem cells and differentiated cells.

Thus, within the scope of the invention are compositions comprising MASCs, e.g. pharmaceutical compositions comprising a therapeutically effective amount of MASCs.

In certain embodiments, the invention further relates to methods of use of the MASCs, e.g., methods of treatment and/or tissue/organ repair by administering MASCs. The mode of administration can be determined by a person of skill in the art depending on the type of organ/injury to be treated. For example, MASCs may be administered by injection (as a suspension) or implanted on a biodegradable matrix.

In one embodiment, MASCs may be used for regeneration and repair of damaged organs or tissues. For example, MASCs (isolated from the same patient or HLA-matched allogenic MASCs) can be seeded into a biocompatible, biodegradable matrix at a density of 1×10⁷ cells per cubic centimeter and cultured undifferentiated in vitro until cell attachment is achieved. This construct of cells+matrix is then implanted at the site of the tissue/organ to be repaired. Examples include, but are not limited to, articular cartilage defects, either partial or full-thickness, meniscus, calvaria, and skin burns. An example of a matrix includes polyglycolic acid mesh. In certain embodiments, MASCs may be pre-treated in vitro with appropriate factors to commit the cells to a particular phenotypic pathway or pathways of the tissue/organ to be repaired. For example, MASCs may be pre-treated with bone morphogenetic protein to differentiate them into an osteogenic lineage for repair of large segmental defects in bone. Other examples of use include forming new breast tissue following mastectomy; repairing kidneys or intestines following trauma or diverticulitis, repairing tendons or ligaments following sports injury, treating spinal cord following trauma. Because of their unique stage, it is expected that the MASCs when used therapeutically, will have the potential to differentiate into any of the 208 tissue types, depending on the local cues and other factors applied to the particular treatment site.

In another embodiment, the invention encompasses systemic distribution of stem cells for diseases that have a deficiency of precursor cells, such as osteoporosis or spinal cord injury. For example, MASCs in suspension may be injected into the organ of interest or into the circulatory system, the number of cells injected being from 10.sup.6 to 10.sup.9 in an appropriate amount of physiological saline. Example of systemic injection for a systemic disease is osteoporosis, where an appropriate amount of the MASCs would distribute to the bone and provide an adequate amount of osteoprogenitor cells.

### Example 3

### Human MASCs Implanted in a Dermal Defect in Rats

This experiment presents data for the ability of human MASCs to differentiate in vivo and regenerate tissues. The experimental design was as follows:

1. Retired male breeder rats were used.

2. 2 cm diameter full-thickness defects were created on the back of the rats. This defect included the epidermis, dermis, and underlying tissue to the underlying skeletal muscle.

3. Each defect was assigned to one of three treatments;
Empty defect

PGA felt alone: 3 cm diameter, 4 mm thick
PGA with MASCs: 60×106 Cells for grown into the polymer for 1 wk

4. All wounds were given standard care With Xeroform

5. Animals euthanized at 8 weeks and the defect dissected and processed for histology and immunohistochemistry.

6. Human MASCs were tracked with an antibody specific for human γ-actin as used (xenogenic injection).

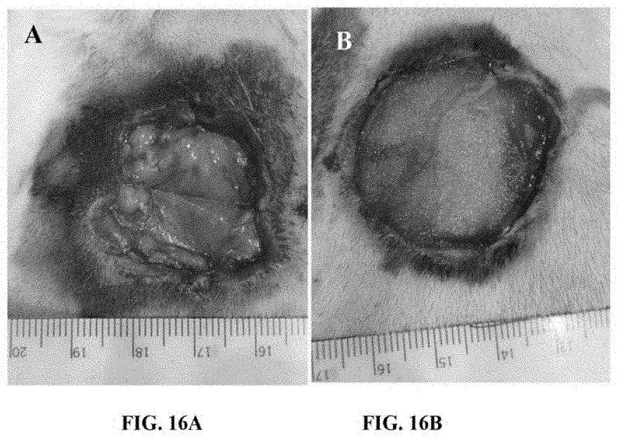

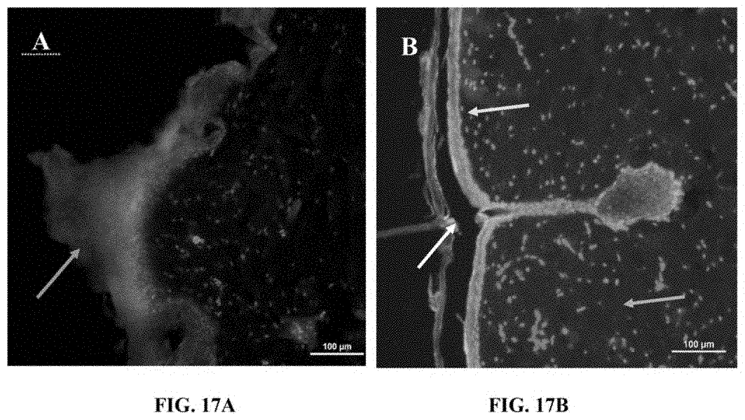

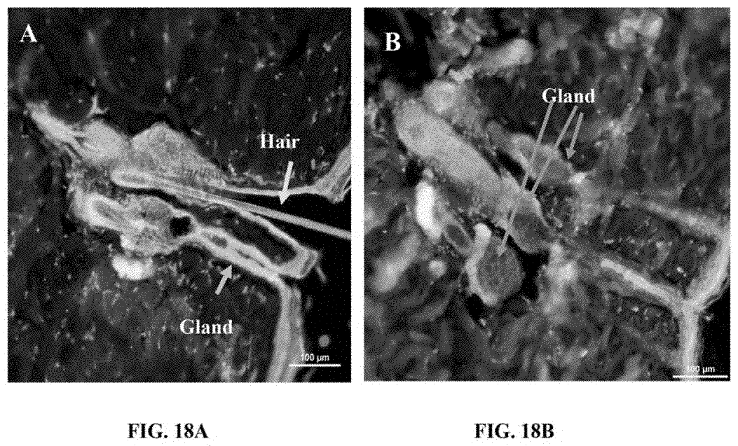

FIGS. 16A-16B show defects immediately post-op. FIGS. 17A-17B show immunohistochemistry 8 weeks post-op. Sections were stained with an antibody to keratinocytes (red), human MASCs (green), and nuclei (blue). FIGS. 18A-18B show immunohistochemistry of the dermal defect treated with PGA+MASCs 8 weeks post-op. FIGS. 19A-19B show the dermal defect treated with PGA+MASCs 8 weeks post-op stained for endothelial cells.

Human MASCs regenerated a full thickness, critical sized defect in the skin of retired breeder rats. This age of rat was chosen because older animals cannot regenerate on their own. This is demonstrated by FIG. 17A, where the empty defect has keratinocytes, but they were not organized into an epidermis. The defect is filled with scar tissue. In contrast, defects treated with PGA+MASCs had normal appearing, epidermis and dermis (FIG. 17B). Immunohistochemical staining for human gamma actin shows that the epidermis and dermis are of human origin, indicating that the human MASCs differentiated to keratinocytes and dermal fibroblasts. Further staining confirms the differentiation to keratinocytes (FIG. 18A) but also shows that the regenerated skin had human MASC-derived eccrine glands (FIGS. 18A and 18B) and human derived blood vessels (FIGS. 19A and 19B). This represents complete regeneration of the skin, including the secondary structures of hair follicles and eccrine glands.

The MASCs were implanted undifferentiated. Immunohistochemical staining shows that the human MASCs differentiated to 4 phenotypes: keratinocytes, fibroblasts, eccrine gland cells, and endothelial cells.

### References

1. Murry, C. E., and Keller, G. (2008). Differentiation of embryonic stem cells to clinically relevant populations: lessons from embryonic development. Cell, 132(4): 661-680.

2. Aoi T., Yae K., Nakagawa M., Ichisaka T., Okita K., Takahashi K., Chiba T., and Yamanaka S. (2008). Generation of pluripotent stem cells from adult mouse liver and stomach cells, Science, 321: 6997.702.

3. Nakagawa M., Koyanagi M., Tanabe K., Takahashi K., Ichisaka T., Aoi T., Okita K., Mochiduki Y., Takizawa N., Yamanaka S. (2008). Generation of induced pluripotent stem cells without Myc from mouse and human fibroblasts. Nat Biotechnol, 26: 101-106.

4. Meirelles, L. D. (2006). Mesenchymal stem cells reside in virtually all post-natal organs and tissues. Journal of Cell Science, 119(11): 2204-2213. doi:10.1242/jcs.02932.

5. Dominici M. et al. Minimal criteria for defining multipotent mesenchyrnal stromal cells. The International Society for Cellular Therapy position statement. Cytotherapy, 8: 315-317 (2006).

6. Friedenstein A. J., Piatetzky-Shapiro & Petrakova K. V. Osteogenesis in transplants of bone marrow cells. J. Embryo. Exp. Morphol., 16: 381-390 (1966).

7. Kern S., Eichler H., Stoeve J., Klüter H. & Bieback. K. Comparative Analysis of Mesenchymal Stem Cells from Bone Marrow, Umbilical Cord Blood, or Adipose Tissue. STEM CELLS, 24: 1294-1301 (2006).

8. Davies O. G., Cooper P. R., Shelton R. M., Smith A. J. & Scheven B. A. A comparison of the in vitro mineralisation and dentinogenic potential of mesenchymal stem cells derived from adipose tissue, bone marrow and dental pulp. J. Bone Miner. Metab., 33: 371-382 (2014).

9. Bianco P., Robey P. G. & Simmons P. J. Mesenchymal stem cells: revisiting history, concepts, and assays. Cell Stem Cell 2, 313-319(2008),

10. Wagner W. et al. (2009). Aging and Replicative Senescence Have Related Effects on Human Stem and Progenitor Cells. PLos ONE doi:10.1371/journal.pone.0005846

11. Hare, I., Gencheva, M., Evans, R., Fortney, J., Piktel, D., Vos, J. A., . . . Gibson, L. F. (2016). In Vitro Expansion of Bone Marrow Derived Mesenchymal Stem Cells Alters DNA Double Strand Break Repair of Etoposide Induced DNA Damage. Stem Cells International, 2016, 8270464. doi.org/1.01155/2016/8270464

12. Lucas P. A., Calcutt A. F. Southerland S. S., Wilson A., Harvey R., Warejcka D., and Young H. E. (1995). A population of cells resident within embryonic and newborn rat skeletal muscle is capable of differentiating into multiple mesodermal phenotypes. Wound Repair and Regeneration, 3: 449-460, 1995.

13. Black, Jessica C., Cumming U., Sullivan A., Huang W. and Lucas P. A. (2016). Whole transcriptomic comparison between multipotent adult stem cells (MASCs) and two families of mesenchymal stem cells (MSCs). Poster. Graduate Student Research Forum, New York Medical College.

14. Lucas P A, Schultz S, Pine S P. “Pluripotent Adult Stem Cells” U.S. Pat. No. 7,259,011 B2 Issued Aug. 21, 2007.

15. B. Roson-Burgo, F. Sanchez-Guijo, C. D. Cañizo, J. D. L. Rivas. (2014), Transcriptomic portrait of human mesenchymal stromal/stem cells isolated from bone marrow and placenta. BMC Genomics 15: 910. dx.doi.org/10.1186/1471-2164-15-910

16. Choi J, Lee S, Mallard W, Clement K et al. A comparison of genetically matched cell lines reveals the equivalence of human iPSCs and ESCs. Nat Biotechnol 2015 November;33(11): 1173-81. PMID: 26501951

17. email.nymc.edu/owa/redir.aspx?C=RA5dGY0fTzcuCcG7tMEZKPgwf09yzW09piBadbR DSM8hqx3msfTUCA..&URL=https%3a%2f%2www.ncbi.nlm.nih.gov%2fbiosample% 3fLinkName%3dbioproject_biosample_a1l%26from_uid%3d²²⁹⁹⁹⁸

18. Young, H. E., Morrison, D. C., Martin, J. D., and Lucas, P. A. Cryopreservation of embryonic chick myogenic lineage-committed stem cells. J. Tiss. Cult. Meth., 13; 275-284, 1991.

19. Young, H. E., Ceballos, E. M., Smith, J. C., Lucas, P. A., and Morrison, D. C. Isolation of embryonic chick myosatellite and pluripotent stem cells. J. Tiss. Cult. Meth., 14: 85-92, 1992.

20. Fu W., Li J., Chen G., Li Q., Tang X., Zhang C. Mesenchymal stem cells derived from peripheral blood retain their pluripotency, but undergo senescence during long-term culture. Tissue Engineering Part C: Methods. 2015;21(10):1088-1097. doi: 10.1089/ten.tec.2014.0595.

21. Schultz S S, Lucas P A. Human stem cells isolated from adult skeletal muscle differentiate into neural phenotypes. J Neurosci Methods, 2006;152(1-2):144-55.

22. Schultz S S, Abraham S, Lucas P A. Stem cells isolated from adult rat muscle differentiate across all three dermal lineages. Wound Repair Regen. 2006;14(2):224-31.

Many modifications and variations of this invention can be made without departing from its spirit and scope, as will be apparent to those skilled in the art. The invention is defined by the terms of the appended claims, along with the Rill scope of equivalents to which such claims are entitled. The specific embodiments described herein, including the following examples, are offered by way of example only, and do not by their details limit the scope of the invention.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general Statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention. In addition to those shown and described herein will become apparent to those skilled in the art from the foregoing description and fall within the scope of the appended claims.

What is claimed is:

1. A method for regenerating or repairing tissue in a patient in need thereof, comprising administering a pharmaceutical composition comprising multipotent adult stem cells (MASCs) to the patient, wherein the MASCs express epiregulin (EREG) and do not express OCT4, Sox-2, and Nanog.

2. The method of claim 1, wherein the tissue comprises bone, meniscus, cartilage, skin or any combination thereof.

3. The method of claim 2, wherein the tissue comprises skin.

4. The method of claim 1, wherein the wherein the MASCs are derived from human foreskin, adult skin, skeletal muscle, adipose tissue, bone marrow, or any combination thereof.

5. The method of claim 4, wherein the wherein the MASCs are derived from human foreskin.

6. The method of claim 1, wherein the pharmaceutical composition further comprises a biodegradable matrix.

7. The method of claim 6, wherein the biodegradable matrix comprises a synthetic polymer.

8. The method of claim 7, wherein the synthetic polymer comprises a hydroxyl acid.

9. The method of claim 8, wherein the hydroxyl acid is polyglycolic acid.

10. The method of claim 1, wherein the MASCs do not form teratomas in vivo.

11. The method of claim 1, wherein the MASCs differentiate into mesodermal cells, ectodermal cells, endodermal cells, or any combination thereof.

12. The method of claim 6, wherein the pharmaceutical composition comprises at least about 20 million MASCs per cubic centimeter of the biodegradable matrix.
